(19) United States
(12) Patent Application Publication
(10) Pub. No.: US 2015/0196280 A1
(43) Pub. Date: Jul. 16, 2015

(54) ULTRASOUND DIAGNOSTIC APPARATUS, ULTRASOUND IMAGE GENERATING METHOD, AND RECORDING MEDIUM

(71) Applicant: FUJIFILM Corporation, Tokyo (JP)

(72) Inventor: Hiroaki YAMAMOTO, Ashigara-kami-gun (JP)

(73) Assignee: FUJIFILM Corporation, Tokyo (JP)

(21) Appl. No.: 14/669,889

(22) Filed: Mar. 26, 2015

Related U.S. Application Data
(63) Continuation of application No. PCT/JP2013/075529, filed on Sep. 20, 2013.

(30) Foreign Application Priority Data
Sep. 27, 2012 (JP) 2012-215264
Jul. 11, 2013 (JP) 2013-145443

(51) Int. Cl. A61B 8/08; A61B 8/00; A61B 8/14

(52) U.S. Cl. CPC A61B 8/5207; A61B 8/14; A61B 8/486; A61B 8/4488; A61B 8/4245; A61B 8/465; A61B 8/5269

(57) ABSTRACT

There are provided an ultrasonic diagnostic apparatus, an ultrasound image generating method, and a recording medium having stored therein a program capable of generating an ultrasound image with a precision close to that of multi-focus even with a moving image. In the case of a motion picture photographing mode, transmission/reception is performed with single focus, and multi-line processing is performed based on received element data. Thereafter, image processing is performed, and a moving image of an ultrasonic image is displayed or a sound velocity value is calculated. In the case of a still picture photographing mode, transmission/reception is performed with multi-focus, and phasing addition processing and the like are performed on received element data. Thereafter, image processing is performed, and a still image of an ultrasonic image is displayed or a sound velocity value is calculated.

### CROSS-REFERENCE TO RELATED APPLICATIONS

This application is a Continuation of PCT International Application No. PCT/JP2013/075529 filed on Sep. 20, 2013, which claims priority under 35 U.S.C. §119(a) to Japanese Patent Application No. 2012-215264 filed on Sep. 27, 2012 and Japanese Patent Application No. 2013-145443 filed on Jul. 11, 2013. Each of the above application(s) is hereby expressly incorporated by reference, in its entirety, into the present application.

### BACKGROUND OF THE INVENTION

The present invention relates to an ultrasound diagnostic apparatus, an ultrasound image generating method, and a recording medium having stored therein a program, which generate an ultrasound image used for inspection or diagnosis of an inspection object by performing imaging of the inspection object such as an organ in a living body by the transmission and reception of ultrasonic beams; in particular, to an ultrasound diagnostic apparatus, an ultrasound image generating method, and a recording medium having stored therein a program, which perform multi-line processing with single focus in a motion picture photographing mode and perform normal processing with multi-focus in a still picture photographing mode.

Conventionally, in the medical field, ultrasonic examination apparatuses such as ultrasound image diagnostic apparatuses using ultrasound images are being put into practical use. In general, this type of ultrasonic examination apparatus has an ultrasound probe with a plurality of built-in elements (ultrasound transducers), and an apparatus main body connected with the ultrasound probe, and generates an ultrasound image by transmitting ultrasonic beams from the plurality of elements of the ultrasound probe toward an inspection object (hereinafter, also referred to as a subject), receiving ultrasonic echoes from the subject by the ultrasound probe, and electrically processing the received ultrasonic echo signals in the apparatus main body.

In the ultrasonic examination apparatus, when generating the ultrasound image, ultrasonic beams are transmitted from the plurality of elements of the probe to an inspection object region in a subject, for example, organs in a living body or lesions or the like in the organs, by matching focus points, and ultrasonic echoes from a reflecting body in the inspection object region, for example, the surface or interface of the organs, the lesions, or the like, are received via the plurality of elements. However, since the ultrasonic echoes reflected by the same reflecting body are received by a plurality of elements, with respect to an ultrasonic echo signal reflected by the reflecting body positioned at a focus point position of an ultrasonic beam transmitted from a transmission element and received by the transmission element, an ultrasonic echo signal reflected by the same reflecting body and received by another element which is not the transmission element is delayed. For this reason, element data is generated by analog-to-digital (A/D) converting the ultrasonic echo signals received by the plurality of elements and is subjected to a reception focusing process, that is, delay correction, phase matching and phasing addition, to generate a sound ray signal. An ultrasound image is generated based on the sound ray signal obtained in this manner.

For example, JP 2010-207490 A describes a method of, using respective average sound velocities from two regions of interest ROI1 and ROI2 to an ultrasound probe, i.e., ambient sound velocities, determining an average sound velocity between the two regions of interest, i.e., a local sound velocity.

In JP 2010-207490 A, ROI1 is set in the subject at a distance (depth) d from the ultrasound probe and ROI2 is set in the subject at a distance (depth) d+Δd from the ultrasound probe.

Next, an average sound velocity C₁ on a path from the ultrasound probe to ROI1 is determined based on a set sound velocity value at which the beam focusing degree (the image quality of the ultrasound image) at ROI1 is the maximum, and an average sound velocity C₂ on a path from the ultrasound probe to ROI2 is determined based on a set sound velocity value at which the beam focusing degree (the image quality of the ultrasound image) at ROI2 is the maximum.

Then, an average sound velocity Cₓ on the path from ROI1 to ROI2 is determined based on the average sound velocities C₁ and C₂ and the distances from the ultrasound probe to ROI1 and ROI2. When ROI1 and ROI2 are set above and below the target region in this manner, it is possible to determine the sound velocity in this region.

### SUMMARY OF THE INVENTION

In JP 2010-207490 A described above, in order to accurately determine the average sound velocities on paths from the ultrasound probe to ROI1 and ROI2, it is necessary to obtain a clear reflected wavefront by transmitting transmission beams with their focus points being matched at the respective positions of ROI1 and ROI2. For this reason, the number of times of transmission focusing is increased according to the number of ROIs and, as a result, there is a problem in that the frame rate is decreased, which is not suitable for a moving image.

An object of the present invention is to solve the problems of the related art described above and to provide an ultrasound diagnostic apparatus, an ultrasound image generating method, and a recording medium having stored therein a program capable of generating an ultrasound image with a precision close to that of multi-focus even with a moving image.

In addition, an object of the present invention is to provide an ultrasound diagnostic apparatus, an ultrasound image generating method, and a recording medium having stored therein a program capable of calculating a sound velocity value with a precision close to that of multi-focus even for a moving image.

In order to attain the above objects, the present invention provides as its first aspect an ultrasound diagnostic apparatus inspecting an inspection object using ultrasonic beams, comprising: a probe having a plurality of elements arranged therein, the probe being configured to transmit the ultrasonic beams, receive ultrasonic echoes reflected by the inspection object, and output analog element signals according to the received ultrasonic echoes; a transmitter configured to cause the probe to transmit the ultrasonic beams plural times through the plurality of elements such that predetermined transmission focus points are formed; a receiver configured to receive analog element signals that the plurality of elements output in response to transmission of each of the ultrasonic beams for each of the transmission focus points, and carry out a predetermined process; an analog-to-digital converter configured to analog-to-digital convert the analog element signals processed by the receiver into pieces of first element data which are digital element signals; a first data processor configured to generate a piece of second element data corresponding to one of the pieces of first element data from the pieces of first element data; and a photographing mode switching unit configured to switch a mode between a motion picture photographing mode in which a moving image is taken by generating the ultrasonic beams continuously in terms of time and a still picture photographing mode in which a still image is taken by temporarily generating the ultrasonic beams, wherein when the photographing mode switching unit switches the mode to the motion picture photographing mode, the transmitter forms at least one focus point in the inspection object, and the first data processor processes the pieces of first element data.

For instance, the transmitter transmits the ultrasonic beams plural times while changing an element being center. In addition, for instance, the receiver changes an element being center in response to transmission of each of the ultrasonic beams by the transmitter.

The receiver may carry out reception using same elements as the plurality of elements used by the transmitter.

The first data processor may change a number of the pieces of first element data to be processed when the photographing mode switching unit switches the mode to the motion picture photographing mode.

It is preferable to include an image generator configured to generate display image data based on the piece of second element data; and a monitor configured to display a moving image of an ultrasound image based on the display image data.

It is preferable to include an ambient sound velocity determiner configured to determine an ambient sound velocity in the inspection object, and in this case, the image generator generates display image data using the determined ambient sound velocity, and the monitor displays a moving image of an ultrasound image based on the ambient sound velocity.

It is preferable to include a local sound velocity determiner configured to determine a local sound velocity based on the ambient sound velocity, and in this case, the image generator generates the display image data using the determined local sound velocity, and the monitor displays a moving image of an ultrasound image based on the local sound velocity.

It is preferable to include a sound velocity corrector configured to correct a sound velocity based on the ambient sound velocity to obtain a sound velocity correction value, and in this case, the image generator generates the display image data using the sound velocity correction value, and the monitor displays a moving image of an ultrasound image with a sound velocity having been corrected with the sound velocity correction value.

It is preferable to include a second data processor configured to generate data of one line on an ultrasound image based on one of the pieces of first element data, and in this case, when the photographing mode switching unit switches the mode to the still picture photographing mode, the transmitter forms a plurality of focus points in the inspection object, and the second data processor processes the pieces of first element data.

Preferably, an image generator generates display image data based on data of one line on an ultrasound image generated by the second data processor, and a monitor displays a still image of an ultrasound image based on the display image data.

Preferably, the image generator generates display image data using an ambient sound velocity determined by an ambient sound velocity determiner, and the monitor displays a still image of an ultrasound image based on the ambient sound velocity.

Preferably, a local sound velocity determiner determines a local sound velocity based on the ambient sound velocity, the image generator generates display image data using the determined local sound velocity, and the monitor displays a still image of an ultrasound image based on the local sound velocity.

Preferably, a sound velocity corrector corrects a sound velocity based on the ambient sound velocity to obtain a sound velocity correction value, the image generator generates display image data using the sound velocity correction value, and the monitor displays a still image of an ultrasound image based on the sound velocity correction value.

It is preferable to include an element data retaining unit configured to retain at least either one of the pieces of first element data and the pieces of second element data.

Preferably, the first data processor generates pieces of first reception data by performing phasing addition on the respective pieces of first element data just before generating the piece of second element data from the pieces of first element data, and generates a piece of second reception data corresponding to one of the pieces of first reception data from the pieces of first reception data.

It is preferable to include an image generator configured to generate display image data based on the piece of second reception data; and a monitor configured to display a moving image of an ultrasound image based on the display image data.

Preferably, the first data processor includes a superimposition processor configured to generate the piece of second element data by superimposing two or more of the pieces of first element data based on receiving times when the plurality of elements receive ultrasonic echoes and positions of the plurality of elements.

The present invention provides as its second aspect an ultrasound diagnostic apparatus inspecting an inspection object using ultrasonic beams, comprising: a probe having a plurality of elements arranged therein, the probe being configured to transmit the ultrasonic beams, receive ultrasonic echoes reflected by the inspection object, and output analog element signals according to the received ultrasonic echoes; a transmitter configured to cause the probe to transmit the ultrasonic beams plural times through the plurality of elements such that predetermined transmission focus points are formed; a receiver configured to receive analog element signals that the plurality of elements output in response to transmission of each of the ultrasonic beams for each of the transmission focus points, and carry out a predetermined process; an analog-to-digital converter configured to analog-to-digital convert the analog element signals processed by the receiver into pieces of first element data which are digital element signals; a first data processor configured to carry out a phasing addition process on the pieces of first element data and generate a piece of second element data corresponding to one of the pieces of first element data after phasing addition; and a photographing mode switching unit configured to switch a mode between a motion picture photographing mode in which a moving image is taken by generating the ultrasonic beams continuously in terms of time and a still picture photographing mode in which a still image is taken by temporarily generating the ultrasonic beams, wherein when the photographing mode switching unit switches the mode to the motion picture photographing mode, the transmitter forms at least one focus point in the inspection object, and first data processor processes the pieces of first element data after phasing addition.

The present invention provides as its third aspect an ultrasound image generating method for acquiring an ultrasound image for use in inspecting an inspection object using a probe having a plurality of elements arranged therein, the probe transmitting ultrasonic beams, receiving ultrasonic echoes reflected by the inspection object, and outputting analog element signals according to the received ultrasonic echoes, the method comprising the steps of: when a mode is switchable between a motion picture photographing mode in which a moving image is taken by generating ultrasonic beams continuously in terms of time and a still picture photographing mode in which a still image is taken by temporarily generating the ultrasonic beams, and the mode is switched to the motion picture photographing mode, causing the probe to transmit the ultrasonic beams plural times through the plurality of elements such that predetermined transmission focusing points are formed, while outputting analog element signals that the plurality of elements output in response to transmission of each of the ultrasonic beams; analog-to-digital converting the analog element signals into pieces of first element data which are digital element signals; and generating a piece of second element data corresponding to one of the pieces of first element data from the pieces of first element data, with at least one focus point being formed in the inspection object.

For instance, a plurality of focus points in the inspection object are formed; the ultrasonic beams are transmitted to transmission focus points; the pieces of first element data are obtained; and data of one line on an ultrasound image is generated based on one of the pieces of first element data, when the mode is switched to the still picture photographing mode.

The present invention provides as its fourth aspect a computer readable recording medium having stored therein a program that causes a computer to execute the steps of the ultrasound image generating method of the third aspect of the present invention as a procedure.

According to the ultrasound diagnostic apparatus, the ultrasound image generating method, and the recording medium of the present invention, it is possible to generate an ultrasound image and also calculate a sound velocity value with a precision close to that of multi-focus even for a moving image. The program is also capable of generating an ultrasound image and calculating a sound velocity value with a precision close to that of multi-focus even for a moving image.

### BRIEF DESCRIPTION OF THE DRAWINGS

FIG. 1 is a block diagram illustrating an ultrasound diagnostic apparatus of a first embodiment of the present invention.

FIG. 2 is a conceptual diagram for describing an example of a reception focusing process in the ultrasound diagnostic apparatus depicted in FIG. 1.

FIG. 3 is a block diagram conceptually illustrating an example of a configuration of an element data processor of the ultrasound diagnostic apparatus depicted in FIG. 1.

FIG. 4A and FIG. 4C are conceptual diagrams each for describing transmission and reception of ultrasonic waves using an ideal ultrasonic beam and FIG. 4B and FIG. 4D are conceptual diagrams each showing element data obtained by the transmission and reception of ultrasonic waves.

FIG. 5A and FIG. 5C are conceptual diagrams each for describing the ultrasound transmission and reception with an actual ultrasonic beam and FIG. 5B and FIG. 5D are conceptual diagrams each showing element data obtained by the transmission and reception of ultrasonic waves.

FIG. 6A and FIG. 6B are conceptual diagrams for describing a path of a sound wave in the case where the transmission and reception of ultrasonic waves is performed with respect to the same reflection point using different center elements, FIG. 6C is a conceptual diagram for describing element data obtained by a plurality of elements, and FIG. 6D is a conceptual diagram for describing each of the delay times of the element data depicted in FIG. 6C.

FIGS. 7A to 7C and FIGS. 7D to 7F are conceptual diagrams for describing element data in cases of a true signal and a ghost, respectively, separately showing element data, delay times thereof, and states where the pieces of element data are superimposed, FIG. 7G is a conceptual diagram for describing states where the pieces of element data corresponding to a plurality of elements are superimposed, and FIG. 7H is a conceptual diagram for describing the results of superimposing the pieces of element data in FIG. 7G.

FIG. 8 is a block diagram conceptually illustrating an example of a configuration of a sound velocity determiner of the ultrasound diagnostic apparatus depicted in FIG. 1.

FIG. 9 is a flow chart for describing an example of a sound velocity determining process of the ultrasound diagnostic apparatus depicted in FIG. 1.

FIG. 10 is a flow chart for describing a sound velocity determining method in the flow chart of FIG. 9.

FIG. 11 is a flow chart for describing a still picture photographing mode and a motion picture photographing mode of an ultrasound diagnostic apparatus of a first embodiment of the present invention.

FIG. 12 is a flow chart for describing a sound velocity determining method.

FIG. 13 is a block diagram illustrating an ultrasound diagnostic apparatus of a second embodiment of the present invention.

FIGS. 14A and 14B are schematic diagrams for describing a calculation process of a local sound velocity value.

FIG. 15 is a flow chart for describing an example of the calculation process of the local sound velocity value.

FIG. 16 is a block diagram illustrating an ultrasound diagnostic apparatus of a third embodiment of the present invention.

### DETAILED DESCRIPTION OF THE INVENTION

Detailed description will be given of an ultrasound diagnostic apparatus, an ultrasound image generating method, and a computer readable recording medium having stored therein a program of the present invention based on preferred embodiments illustrated in the attached diagrams.

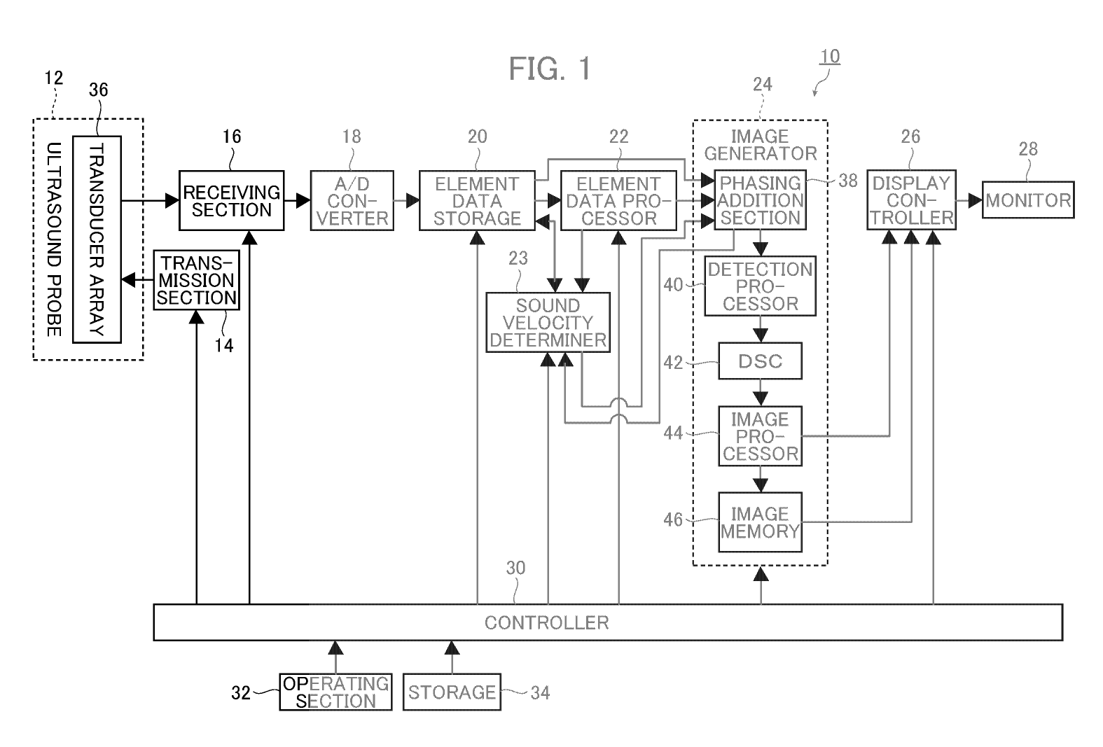

FIG. 1 is a block diagram illustrating an ultrasound diagnostic apparatus of a first embodiment of the present invention.

An ultrasound diagnostic apparatus 10 illustrated in FIG. 1 which will be described in detail below has a motion picture photographing mode in which a moving image is taken by continuously generating ultrasonic beams in time series and a still picture photographing mode in which a still image is taken by temporarily generating ultrasonic beams, and has a photographing mode switching unit for switching the motion picture photographing mode and the still picture photographing mode.

In the case of the motion picture photographing mode, transmission and reception is performed with single focus (one focus point) with respect to a subject (an inspection object), multi-line processing which will be described in detail below is performed based on received element data (first element data), and an ultrasound image (a moving image) is generated and displayed, or calculation of a sound velocity value is carried out. On the other hand, in the case of the still picture photographing mode, similarly to the related art, transmission and reception is performed with multi-focus (a large number of focus points) with respect to a subject, and an ultrasound image (a still image) is generated and displayed, or calculation of a sound velocity value is carried out.

Below, detailed description will be given of an ultrasound diagnostic apparatus 10 which has the motion picture photographing mode and the still picture photographing mode.

As illustrated in FIG. 1, the ultrasound diagnostic apparatus 10 has an ultrasound probe 12, a transmission section 14 and a receiving section 16 connected with the ultrasound probe 12, an analog-to-digital (A/D) converter 18, an element data storage 20, an element data processor 22 (first data processor), a sound velocity determiner 23, an image generator 24, a display controller 26, a monitor 28, a controller 30, an operating section 32, and a storage 34.

In the example in the diagram, the transmission section 14, the receiving section 16, the A/D converter 18, the element data storage 20, the element data processor 22, the sound velocity determiner 23, the image generator 24, the display controller 26, the monitor 28, the controller 30, the operating section 32, and the storage 34 configure the apparatus main body of the ultrasound diagnostic apparatus 10.

The ultrasound probe 12 (hereinafter, referred to as the probe 12) has a transducer array 36 which can be used in a normal ultrasound diagnostic apparatus.

The transducer array 36 has a plurality of elements, that is, ultrasound transducers, which are one-dimensionally or two-dimensionally arranged in an array. When taking an ultrasound image of a subject (an inspection object), these ultrasound transducers transmit ultrasonic beams to the subject in accordance with driving signals respectively supplied from the transmission section 14, receive ultrasonic echoes from the subject, and output reception signals. In the present embodiment, each of a predetermined number of the ultrasound transducers which form one set out of the plurality of ultrasound transducers of the transducer array 36 generates each component of one ultrasonic beam, and one set of the predetermined number of ultrasound transducers generates one ultrasonic beam to be transmitted to the subject.

Each of the ultrasound transducers is configured of oscillators formed with electrodes at both ends of a piezoelectric body formed of, for example, a piezoelectric ceramic represented by lead zirconate titanate (PZT), a piezoelectric polymer represented by polyvinylidene fluoride (PVDF), a piezoelectric single crystal represented by lead magnesium niobate-lead titanate solid solution (PMN-PT), or the like.

When a pulsed or continuous wave voltage is applied to the electrodes of the oscillator, the piezoelectric body expands and contracts according to the applied voltage, and pulsed or continuous wave ultrasonic waves are generated from each oscillator. In addition, the ultrasonic waves generated from the oscillators converge to be combined (that is, transmission focusing is performed on the ultrasonic waves) at a set focus point according to driving delays of the respective oscillators, thereby forming an ultrasonic beam.

In addition, the oscillators expand and contract in response to entered ultrasonic echoes reflected inside the subject and generate electric signals according to the size of the expansion and contraction. The electric signals are output to the receiving section 16 as the reception signals.

The transmission section 14 includes, for example, a plurality of pulsars and supplies a driving signal (applies a driving voltage) to each of the ultrasound transducers (oscillators) of the probe 12.

For example, in accordance with a sound velocity or with a sound velocity distribution set based on a transmission delay pattern selected according to a control signal from the controller 30, driving signals are supplied to a plurality of ultrasound transducers (hereinafter, referred to as ultrasound elements) forming one set with delay amounts of the respective driving signals being adjusted such that the ultrasonic beam components which are transmitted from one set of a predetermined number of ultrasound elements in the transducer array 36 form one ultrasonic beam.

Furthermore, transmission focusing for adjusting a delay amount of a driving signal (an application timing of a driving voltage) is performed such that the ultrasonic waves transmitted by the plurality of ultrasound transducers form a desired ultrasonic beam that is to converge at a predetermined focus point (transmission focusing point) set in the subject, and the driving signal is supplied to the ultrasound transducers. It is possible to set a large number of focus points in the depth direction of the subject.

Here, the transmission delay pattern may be corrected according to an ambient sound velocity value, a local sound velocity value, and a sound velocity correction value to be described below. In this manner, the desired ultrasonic beam is transmitted from the probe 12 (the transducer array 36) to the subject. Here, the transmission section 14 and the controller 30 configure a focus controller.

According to a control signal from the controller 30, the receiving section 16 receives ultrasonic echoes from the subject, which are generated by the interaction between the ultrasonic beam and the subject using each of the ultrasound elements of the transducer array 36, amplifies and outputs a reception signal, that is, an analog element signal associated with each of the ultrasound elements, and supplies the amplified analog element signal to the A/D converter 18.

The method of transmitting and receiving the ultrasonic waves in the ultrasound diagnostic apparatus 10 of the present invention is basically the same as that of a known ultrasound diagnostic apparatus.

Accordingly, in a single transmission and reception of ultrasonic waves (the transmission of one ultrasonic beam and the reception of ultrasonic echoes corresponding to this transmission), neither the number of ultrasound transducers (the number of transmission openings) which generate the ultrasonic waves nor the number of ultrasound transducers (the number of reception openings) which receive the ultrasonic waves (through which the receiving section 16 receives the reception signal) is limited as long as they are plural. In addition, in a single transmission and reception, the number of openings may be the same or different between the transmission and the reception.

In addition, with adjacent ultrasonic beams in at least the azimuth direction (the arrangement direction of the ultrasound transducers), as long as transmission regions overlap, neither the number of times of the transmission and reception of the ultrasonic waves (number of sound rays) for forming one ultrasound image nor the intervals of the ultrasound transducers (center elements) being the center of the transmission and reception (that is, the density of the scanning lines) is limited. Accordingly, the transmission and reception of the ultrasonic waves may be performed with all of the ultrasound transducers corresponding to the region scanned with ultrasonic waves as the center elements, or the transmission and reception of the ultrasonic waves may be performed with ultrasound transducers at predetermined intervals, such as intervals of every two transducers or every four transducers, as the center elements. The receiving section 16 may change the element assuming the center to correspond to the transmission of the ultrasonic beams by the transmission section 14.

The A/D converter 18 is connected with the receiving section 16 and A/D converts the analog reception signal supplied from the receiving section 16 into element data (first element data) which is a digital reception signal. The A/D converter 18 supplies the A/D converted element data to the element data storage 20.

The element data storage 20 sequentially stores the element data supplied from the A/D converter 18. In addition, the element data storage 20 stores information relating to the frame rate (for example, the depth of the reflecting position of the ultrasonic waves, the density of the scanning lines, or a parameter indicating a visual field width) input from the controller 30 in association with each piece of element data.

Preferably, the element data storage 20 stores all the pieces of element data corresponding to at least one ultrasound image (an ultrasound image of one frame) and does not erase the element data of the ultrasound image before display or during display at least until the display of the ultrasound image is finished.

In the motion picture photographing mode, the controller 30 controls so that the A/D converted element data is output from the element data storage 20 to the element data processor 22.

On the other hand, in the still picture photographing mode, the controller 30 controls so that the A/D converted element data is not output from the element data storage 20 to the element data processor 22, but is output to the sound velocity determiner 23 and the image generator 24 (a phasing addition section 38).

The element data processor 22 is a feature of the present invention and, in the motion picture photographing mode, generates processed element data (second element data) corresponding to each piece of element data by superimposing pieces of element data.

Specifically, under the control of the controller 30, the element data processor 22 superimposes, out of pieces of element data stored in the element data storage 20, certain pieces of element data obtained by a predetermined number (a plurality) of ultrasonic beam transmissions in which the ultrasound transducers assuming the center (the elements assuming the center (center elements)) are different and the transmission regions of the ultrasonic beams overlap, according to the times at which the ultrasound transducers receive the ultrasonic echoes and the positions of the ultrasound transducers, thereby generating processed element data corresponding to the element data (element data of an element of interest to be described below). The element data processor 22 outputs the generated processed element data to the sound velocity determiner 23 and the image generator 24.

In the motion picture photographing mode, the sound velocity determiner 23 determines a sound velocity (ambient sound velocity) of the ultrasonic waves in a subject using the processed element data generated by the element data processor 22. In the still picture photographing mode, the sound velocity determiner 23 determines a sound velocity (ambient sound velocity) of the ultrasonic waves in a subject using the A/D converted element data in the element data storage 20.

Detailed description will be given below of the element data processor 22, the processed element data, the sound velocity determiner 23, and the ambient sound velocity.

Under the control of the controller 30, the image generator 24 generates reception data (a sound ray signal) from the element data (the first element data) supplied from the element data storage 20 or the processed element data (second element data) supplied from the element data processor 22, and generates an ultrasound image from this reception data.

An ultrasound image of a still image is generated from the element data supplied from the element data storage 20, and an ultrasound image of a moving image is generated from the processed element data supplied from the element data processor 22.

The image generator 24 has the phasing addition section 38, a detection processor 40, a DSC 42, an image processor 44, and an image memory 46.

The phasing addition section 38 is connected with the element data storage 20, the element data processor 22, and the sound velocity determiner 23 and, in the motion picture photographing mode, carries out phasing addition on the processed element data generated by the element data processor 22, thereby performing a reception focusing process and generating reception data.

The distance to one reflection point in the subject is different among the ultrasound transducers. Therefore, even with ultrasonic echoes reflected at the same reflection point, the time taken for the ultrasonic echo to arrive at each of the ultrasound transducers is different. According to a reception delay pattern selected by the controller 30, the phasing addition section 38 delays each piece of reception data by an amount corresponding to the difference in the arrival time (the delay time) of the ultrasonic echoes for each of the ultrasound transducers, and carries out phasing addition on the reception data to which the delay time is applied, thereby digitally performing a reception focusing process and generating reception data. The phasing addition section 38 supplies the generated reception data to the detection processor 40.

In addition, in the still picture photographing mode, a plurality of focus points are set in the depth direction in the subject, and the phasing addition section 38 carries out phasing addition on the element data of one element, thereby performing the reception focusing process to generate the reception data, thus generating reception data of one line of the ultrasound image for each of the focus points. At this time, the phasing addition section 38 functions as a second data processor.

Regardless of the motion picture photographing mode or the still picture photographing mode, in the case where the sound velocity (the ambient sound velocity) of the ultrasonic waves in the subject has been determined by the sound velocity determiner 23 and supplied to the phasing addition section 38, the phasing addition section 38 performs the reception focusing process by correcting the delay time, the reception delay pattern, or the like using the ambient sound velocity.

In the case where the ambient sound velocity has not been determined, regardless of the motion picture photographing mode or the still picture photographing mode, the phasing addition section 38 performs the reception focusing process by a known method using a reception delay pattern as described above.

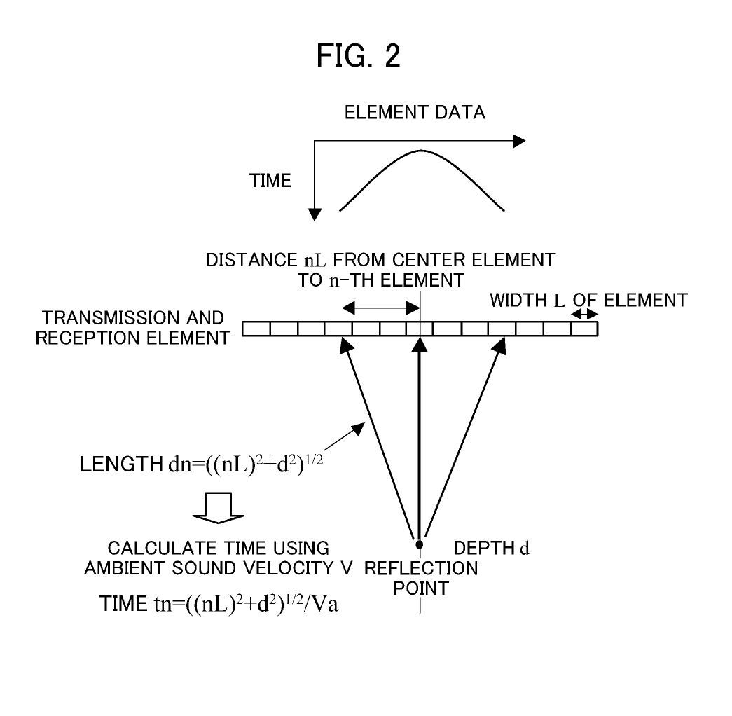

FIG. 2 shows an example of the reception focusing process using the ambient sound velocity.

FIG. 2 shows a case of a linear probe in which the plurality of ultrasound transducers of the probe 12 are arranged in a row in the left and right direction in the diagram. However, the concept may be similarly applied even in the case of a convex probe where only the probe shape is different.

When the width of each of the ultrasound transducers in the azimuth direction is assumed to be L, the distance up to the n-th ultrasound transducer from the ultrasound transducer in the center in the azimuth direction toward the end is nL.

As shown in the diagram, when the reflection point of the ultrasonic wave is assumed to be at a distance (depth) d, which is positioned to be perpendicular to the arrangement direction, from the center ultrasound transducer, the distance (length) dₙ between the n-th ultrasound transducer and the reflection point is calculated using the formula (1).

dₙ=((nL)²+d²)^{1/2} (1)

Accordingly, using the ambient sound velocity V, a time tₙ for the ultrasonic echo from the reflection point to arrive at (be received by) the n-th ultrasound transducer is calculated using the formula (2).

tₙ=dₙ/V=((nL)²+d²)^{1/2}/V (2)

As described above, the distance between each ultrasound transducer and the reflection point is different among the ultrasound transducers. In the case of this example, as shown in the graph at the top of the diagram, the arrival time tₙ of the ultrasonic echo is longer as the ultrasound transducer is positioned closer to the end in the arrangement direction.

Specifically, when the time until the ultrasonic wave is received by the center ultrasound transducer from the reflection point is assumed to be t₁, the ultrasonic wave received by the n-th ultrasound transducer is delayed by the time Δt=tₙ−t₁ with respect to the ultrasonic wave received by the center ultrasound transducer. In the present example, the delay time Δt is a reception delay pattern.

The phasing addition section 38 performs phasing addition on the reception data corresponding to each of the ultrasound transducers using the delay time represented by the time Δt described above and performs a reception focusing process.

In the present invention, the reception focusing process according to the ambient sound velocity is not limited to this method and it is possible to use various known methods.

For example, the controller 30 may select a reception delay pattern according to the ambient sound velocity and supply the control signal according thereto to the phasing addition section 38. Alternatively, the controller 30 may correct the reception delay pattern according to the ambient sound velocity and supply the control signal according to the corrected reception delay pattern to the phasing addition section 38. Alternatively, the phasing addition section 38 may correct the control signal supplied from the controller 30 according to the ambient sound velocity and perform the reception focusing process.

After carrying out correction of the attenuation caused due to the distance according to the depth of the reflection position of the ultrasonic wave on the reception data generated by the phasing addition section 38, the detection processor 40 generates B-mode image data (display image data) which is tomographic image information (brightness image information) in the subject by carrying out an envelope detection process.

The digital scan converter (DSC) 42 converts (raster converts) the B-mode image data generated by the detection processor 40 into image data corresponding to a normal television signal scanning system.

The image processor 44 carries out various types of necessary image processing such as a gradation process on the B-mode image data input from the DSC 42 to generate B-mode image data for display. The image processor 44 outputs the image processed B-mode image data to the display controller 26 for display and stores the image processed B-mode image data in the image memory 46. The image processed B-mode image data is not necessarily stored in the image memory 46.

The image memory 46 is a known storage (a storage medium) which stores the B-mode image data (display image data) processed by the image processor 44. The B-mode image data stored in the image memory 46 is read out to the display controller 26 for display on the monitor 28 as necessary.

The display controller 26 uses the B-mode image data on which predetermined image processing has been carried out by the image processor 44 to display an ultrasound image of a moving image or an ultrasound image of a still image on the monitor 28. The monitor 28 includes, for example, a display device such as a liquid crystal display (LCD) and displays an ultrasound image of a moving image or an ultrasound image of a still image under the control of the display controller 26.

The controller 30 controls each section of the ultrasound diagnostic apparatus 10 on the basis of instructions input from the operating section 32 by an operator.

In addition, the controller 30 supplies various types of information input by an operator via the operating section 32 to necessary units. For example, in the case where information necessary for calculating the delay time used in the element data processor 22 and the phasing addition section 38 of the image generator 24 and information necessary for element data processing in the element data processor 22 are input by the operating section 32, the information is supplied to the transmission section 14, the receiving section 16, the element data storage 20, the element data processor 22, the image generator 24, and the display controller 26 as necessary.

The operating section 32 is used by the operator to perform an input operation and can be formed of a keyboard, a mouse, a trackball, a touch panel, or the like.

In addition, the operating section 32 is provided with an input function for the operator to input various types of information as necessary. For example, the operating section 32 has an input function for inputting information on the probe 12 (the ultrasound transducer); information relating to the generation of processed element data such as the transmission openings and reception openings in the probe 12 (transducer array), the number of pieces of element data to be superimposed and the method; the focus point position of the ultrasonic beam; and the like.

These are input in accordance with, for example, selection of a photograph site (examination site), selection of image quality, selection of the depth of the ultrasound image to be photographed, or the like.

Furthermore, the operating section 32 includes a freeze button for setting a mode of the ultrasound diagnostic apparatus 10 to the motion picture photographing mode or the still picture photographing mode, and the operating section 32 functions as the photographing mode switching unit. When the freeze button is operated, a setting signal which switches the mode from the motion picture photographing mode to the still picture photographing mode is transmitted to the controller 30 to switch the mode from the motion picture photographing mode to the still picture photographing mode. On the other hand, when the operation of the freeze button is released, the mode is switched from the still picture photographing mode to the motion picture photographing mode. Here, the photographing mode switching unit is not limited to the freeze button and a photographing mode switching section which switches the photographing mode described above may be provided.

The storage 34 stores information necessary for the controller 30 to operate and control the ultrasound diagnostic apparatus, such as an operation program for the controller 30 to execute control of each section of the ultrasound diagnostic apparatus 10, the transmission delay pattern and the reception delay pattern, information relating to the generation of processed element data, information on the probe 12 input from the operating section 32, and information on the transmission openings, the reception openings, and the focus point position.

For the storage 34, it is possible to use a known recording medium such as a hard disk, a flexible disk, a magneto-optical disk (MO), a magnetic tape (MT), a random access memory (RAM), a compact disc read only memory (CD-ROM), or a digital versatile disk read only memory (DVD-ROM).

In the ultrasound diagnostic apparatus 10, the element data processor 22, the sound velocity determiner 23, the phasing addition section 38, the detection processor 40, the DSC 42, the image processor 44, the display controller 26, and the like are configured by a central processing unit (CPU) and an operation program causing the CPU to execute various processes. However, in the present invention, these units may be configured by a digital circuit.

As described above, the element data processor 22 generates processed element data by superimposing, out of the pieces of element data (unprocessed element data) stored in the element data storage 20, certain pieces of element data obtained by a predetermined number (a plurality) of ultrasonic beam transmissions in which the ultrasound transducers assuming the center (the center elements) are different and the transmission regions of the ultrasonic beams overlap, according to the receiving times of the ultrasound transducers and the positions of the ultrasound transducers.

In the following description, the ultrasound transducers are also referred to simply as “elements”.

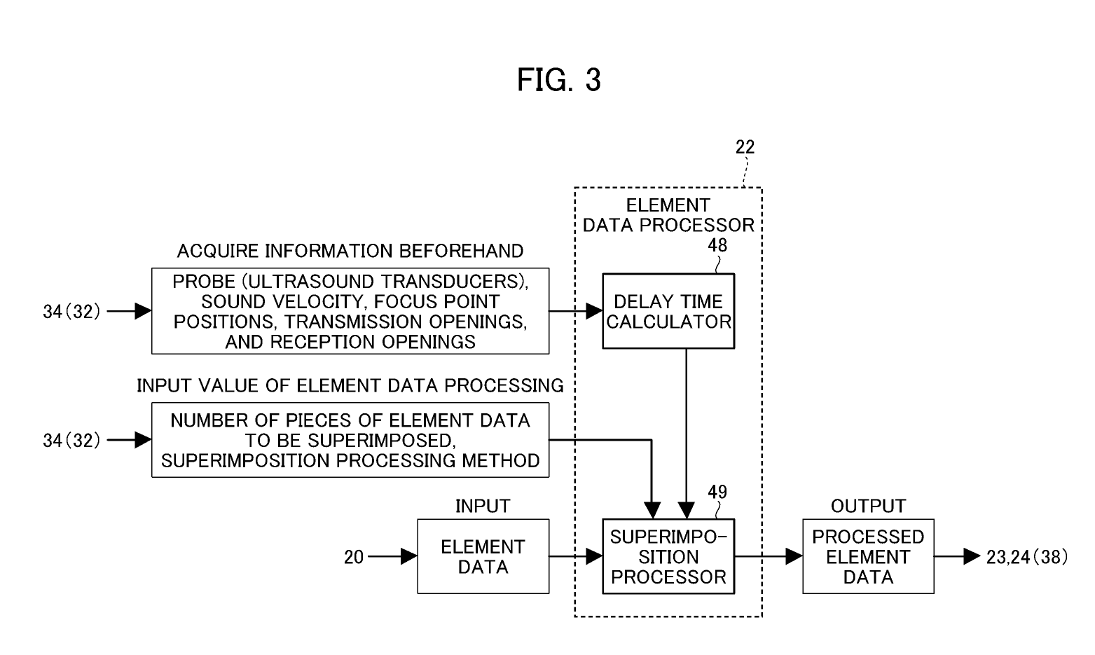

FIG. 3 is a block diagram conceptually illustrating the configuration of the element data processor 22.

As illustrated in FIG. 3, the element data processor 22 has a delay time calculator 48 and a superimposition processor 49.

The delay time calculator 48 acquires beforehand necessary information input from the operating section 32 or stored in the storage 34 after being input from the operating section 32 relating to the probe 12 (the ultrasound transducers (elements)), focus point positions of the ultrasonic beams, the transmission openings and the reception openings of the probe 12, and the like.

In addition, the delay time calculator 48 calculates the delay time of the ultrasonic echoes received by the elements of the reception openings, that is, the element data, based on the geometric positions of the elements of the transmission openings which oscillate the ultrasonic waves in order to transmit (generate) the ultrasonic beams and the elements of the reception openings which receive the ultrasonic echoes from the subject.

The superimposition processor 49 reads out certain pieces of element data (element data obtained with ultrasonic beams where the center elements are different and the transmission regions overlap (two or more pieces of element data generated for two or more target regions)) to be superimposed from the pieces of element data stored in the element data storage 20 based on information relating to the number of pieces of element data to be superimposed and the element data process such as a superimposition processing method as input from the operating section 32 or stored in the storage 34 after being input from the operating section 32.

Furthermore, based on the delay time corresponding to each piece of the element data calculated by the delay time calculator 48, the superimposition processor 49 superimposes two or more pieces of element data according to the reception time, that is, by matching the time and by matching the absolute positions of the receiving elements of the probe, thereby generating the processed element data.

Detailed description will be given of the element data processing performed in the element data processor 22.

Firstly, description will be given of a relationship between ultrasonic beams from the transmission elements and element data obtained by the reception elements in the case where, in the ultrasound probe 12, the ultrasonic beams are transmitted to the subject from the transmission openings, that is, the elements (hereinafter, simply referred to as the transmission elements) which send out the ultrasonic waves in order to transmit the ultrasonic beams, and the element data is obtained by receiving the ultrasonic echoes generated by interaction with the subject at the reception openings, that is, at the elements (hereinafter, simply referred to as the reception elements) which receive the ultrasonic echoes.

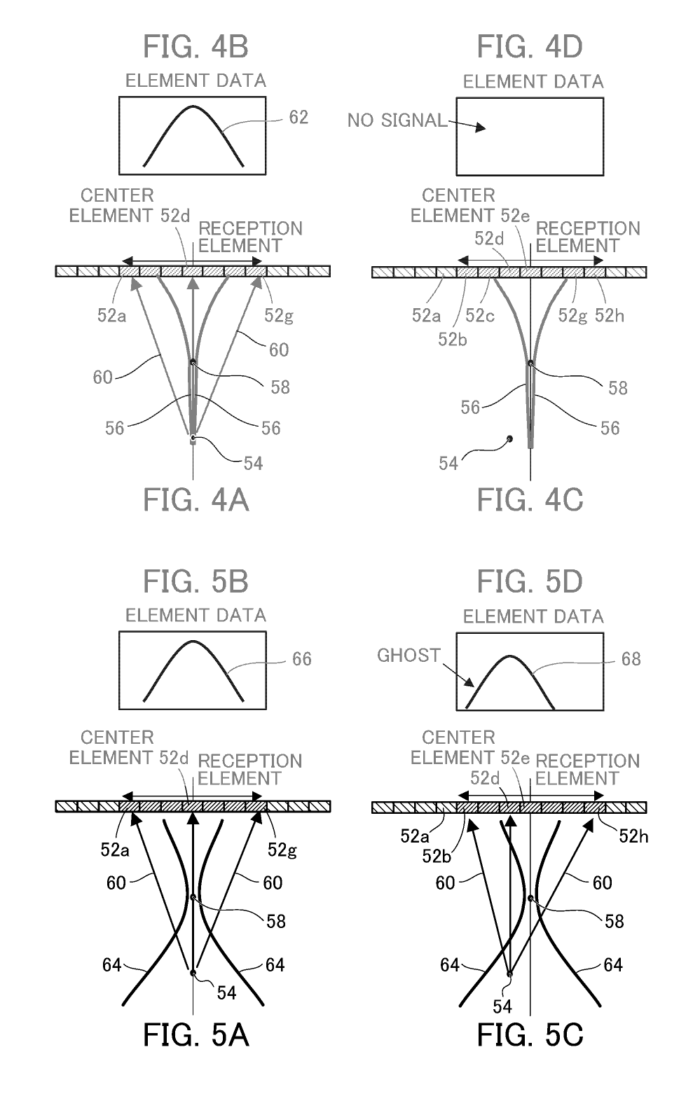

As an example, as shown in FIG. 4A, an ultrasonic beam is transmitted by the transmission section 14 with three elements 52c to 52e as the transmission elements and ultrasonic echoes are received with seven elements 52a to 52g as the reception elements. Next, as shown in FIG. 4C, the ultrasonic beam is transmitted with three elements 52d to 52f as transmission elements by moving (hereinafter, also referred to as shifting) the elements by one element in the azimuth direction and ultrasonic echoes are received by the receiving section 16 with seven elements 52b to 52h as the reception elements to acquire the respective piece of element data.

That is, the center element (the element in the center) is the element 52d in the example shown in FIG. 4A and the center element is the element 52e in the example shown in FIG. 4B.

Now, an ideal case will be considered in which an ultrasonic beam 56 transmitted to the inspection object region including a reflection point 54 is converged at a focus point 58 and narrowed to the element interval or less.

As shown in FIG. 4A, when the ultrasonic beam 56 is transmitted from the elements 52c to 52e which are transmission elements with the element 52d directly above the reflection point 54 (on a straight line linking the reflection point and the focus point) as the center element, and pieces of element data are acquired by receiving the ultrasonic echoes at the elements 52a to 52g which are the reception elements, the focus point 58 of the ultrasonic beam 56 is on a straight line linking the element 52d which is the center element and the reflection point 54. In such a case, since the ultrasonic beam 56 is transmitted up to the reflection point 54, the ultrasonic echoes reflected from the reflection point 54 are generated.

The ultrasonic echoes from the reflection point 54 are received at the elements 52a to 52g which are the reception elements after passing through a receiving path 60 broadening at a predetermined angle, and the element data 62 as shown in FIG. 4B is obtained by the elements 52a to 52g. In FIG. 4B, the vertical axis represents the time and the horizontal axis represents the position (the position of the elements) in the azimuth direction corresponding to FIG. 4A (the same applies to FIG. 4D).

In contrast, as shown in FIG. 4C, in the case where the center element is shifted by the amount of one element, the element 52e next to the element 52d directly above the reflection point 54 becomes the center element.

The ultrasonic beam 56 is transmitted from the elements 52d to 52f which are transmission elements with the element 52e as the center element and the ultrasonic echoes are received at the elements 52b to 52h which are the reception elements. At this time, when the ultrasonic beam 56 is ideal in the same manner, the reflection point 54 is not present in the transmission direction of the ultrasonic beam 56, that is, on a straight line linking the center element 52e and the focus point 58. Accordingly, the ultrasonic beam 56 is not transmitted to the reflection point 54.

Therefore, the ultrasonic echoes reflected from the reflection point 54 are not generated and the elements 52b to 52h which are reception elements do not receive the ultrasonic echoes, and thus, as shown in FIG. 4D, the reflected signal from the reflection point 54 is not obtained (the signal intensity of the element data is “0”).

However, since the actual ultrasonic beam is diffused after being converged at the focus point 58 as an ultrasonic beam 64 shown in FIGS. 5A and 5C, the width is wider than the element interval.

Here, similarly to FIG. 4A, in the case where the ultrasonic beam 64 is transmitted with the elements 52c to 52e as the transmission elements and the element 52d directly above the reflection point 54 as the center element as in FIG. 5A, even when the ultrasonic beam 64 is wide, the focus point 58 is on a straight line linking the element 52d and the reflection point 54. Accordingly, the ultrasonic beam 64 is reflected at the reflection point 54 and ultrasonic echoes are generated.

As a result, in the same manner as the case of FIG. 4A, the ultrasonic echoes from the reflection point 54 are received at the elements 52a to 52g which are the reception elements after passing through a receiving path 60 which broadens at a predetermined angle, and, similarly, true element data 66 as shown in FIG. 5B is obtained.

Next, in the same manner as FIG. 4C, as shown in FIG. 5C, the ultrasonic beam 56 is transmitted by shifting the center element by one element, i.e., with the adjacent element 52e as the center element and the elements 52d to 52f as the transmission elements, and the ultrasonic echoes are received with the elements 52b to 52h as the reception elements. Even in such a case, since the ultrasonic beam 64 is wide, even when the reflection point 54 is not present in the transmission direction of the ultrasonic waves, that is, on a straight line linking the element 52e which is the center element and the focus point 58, the ultrasonic beam 64 is transmitted to (arrives at) the reflection point 54.

Therefore, ultrasonic echoes which do not exist originally or so-called ghost reflected echoes are generated from the reflection point 54 in the transmission direction of the ultrasonic beam. The ghost reflected echoes from the reflection point 54 are received at the elements 52b to 52h which are reception elements after passing through the receiving path 60 which broadens at a predetermined angle as shown in FIG. 5C. As a result, ghost element data 68 as shown in FIG. 5D is obtained by the elements 52b to 52h.

The ghost element data 68 as described above decreases the precision of the ultrasound image generated from the element data.

The element data processor 22 calculates the delay time corresponding to the element data in the delay time calculator 48, and the superimposition processor 49 superimposes two or more pieces of element data according to the delay time and the absolute positions of the elements, whereby the true element data is emphasized and the ghost element data is attenuated to generate the processed element data which is element data with high precision.

As described above, the delay time calculator 48 calculates the delay time of the element data received at each of the elements of the reception elements (reception openings).

That is, the propagation distance of the ultrasonic beam 64 shown in FIG. 5C is the sum of the transmission path where the ultrasonic beam 64 reaches the reflection point 54 from the center element 52e via the focus point 58 and the receiving path where the ghost reflected echoes from the reflection point 54 reach each of the elements 52b to 52h which are the reception elements.

The propagation distance of the ultrasonic beam 64 shown in FIG. 5C is longer than the propagation distance of the ultrasonic beam 64 shown in FIG. 5A, that is, the sum of the transmission path where the ultrasonic beam 64 reaches the reflection point 54 from the center element 52d via the focus point 58 and the receiving path where the true reflected echoes from the reflection point 54 reach the elements 52a to 52g which are the reception elements.

Therefore, the ghost element data 68 as shown in FIG. 5D is delayed compared to the true element data 66 as shown in FIG. 5B.

In the delay time calculator 48 of the element data processor 22, the time difference between the true element data and the ghost element data, that is, the delay time of the ghost element data is calculated from the sound velocity, the transmission elements, the focus point of the ultrasonic beam, the reflection point of the subject, and the geometric arrangement of the reception elements.

Accordingly, in the calculation of the delay time, information on the shape of the probe 12 (the element interval, the probe type such as linear, convex, or the like), the sound velocity, the position of the focus point, the transmission opening, the reception opening, and the like is necessary. In the delay time calculator 48, the information input by the operating section 32 or stored in the storage 34 is acquired to calculate the delay time. For the sound velocity, use may be made of a fixed value (for example, 1540 m/sec) set in advance, a sound velocity (an ambient sound velocity) determined by the sound velocity determiner to be described below, or one input by the operator.

It is possible to calculate the delay time from the difference in the propagation time calculated using the sound velocity and the total length (propagation distance) of the transmission path of the ultrasonic beam from the transmission element to the reflection point via the focus point and the receiving path of true reflected ultrasonic echoes or ghost reflected signals from the reflection point up to the reception elements, the total length being calculated from the geometric arrangement of, for example, the transmission elements, the focus point of the ultrasonic beam, the reflection point in the subject, and the reception elements.

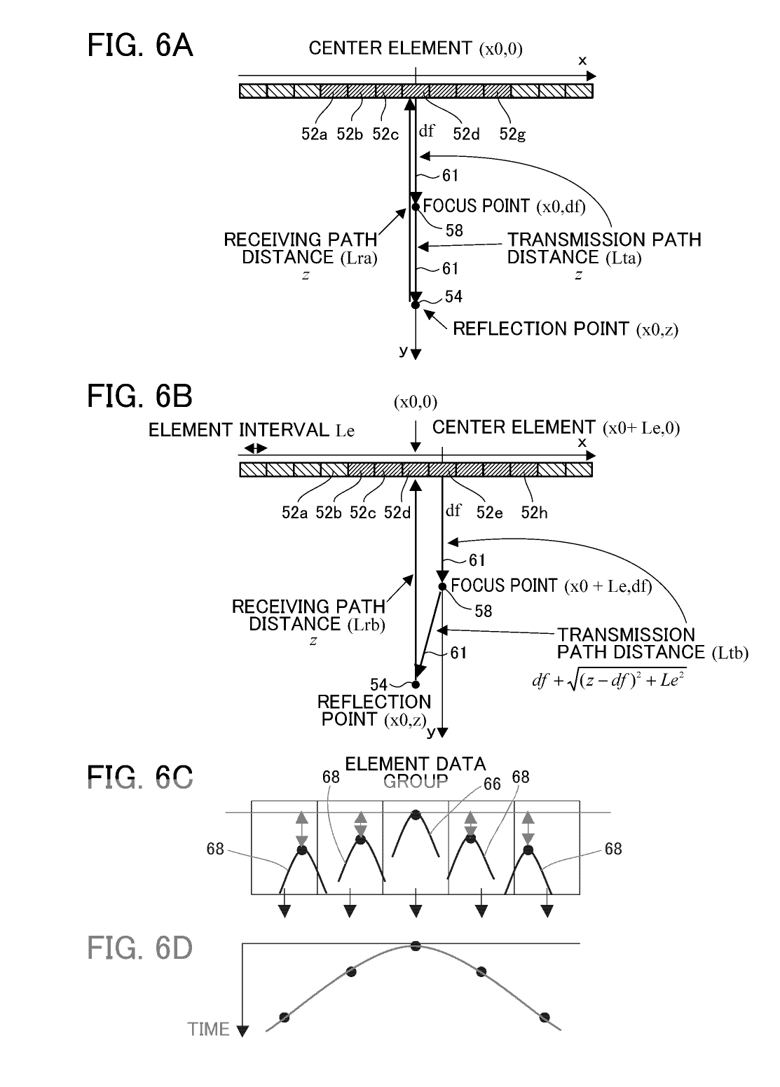

In the present invention, for example, as shown in FIG. 6A and FIG. 6B, it is possible to determine the length of the transmission path and the receiving path of the ultrasonic beam in the case of the true ultrasonic echoes and the ghost reflected echoes. Here, in FIGS. 6A and 6B, the x direction is the azimuth direction and the y direction is the depth direction.

In addition, in FIG. 6A, the transmission and reception of the ultrasonic waves is performed in the same manner as in FIG. 5A and, in FIG. 6B, the transmission and reception of the ultrasonic waves is performed in the same manner as in FIG. 5C.

In the case of the true ultrasonic echoes, as shown in FIG. 6A (FIG. 5A), the element 52d which is the center element, the focus point 58, and the reflection point 54 are all positioned on the same line in the azimuth direction. That is, the focus point 58 and the reflection point 54 are positioned directly below the center element 52d.

Accordingly, when the position of the element 52d which is the center element is assumed to be coordinates (x0, 0) which are two-dimensional x-y coordinates, the x coordinates of the focus point 58 and the reflection point 54 are also “x0”. Below, the position of the focus point 58 in the transmission is coordinates (x0, df), the position of the reflection point 54 is coordinates (x0, z), and the interval of the elements is Le.

At this time, the length (transmission path distance) Lta of a transmission path 61 of the ultrasonic beam from the element 52d which is the center element to the reflection point 54 via the focus point 58 and the length (the receiving path distance) Lra of the receiving path 60 of the true reflected ultrasonic echoes from the reflection point 54 to the element 52d can be calculated using Lta=Lra=z.

Accordingly, in the case of the true ultrasonic echoes, the propagation distance Lua of the ultrasonic echoes is Lua=Lta+Lra=2z.

Next, as shown in FIG. 6B, by shifting the transmitting element and the reception element by one element in the x direction (the azimuth direction) (shifting in the direction to the right in the diagram), transmission and reception are performed with the center element set to the element 52e. As shown in FIG. 5C, in this case, the echoes reflected at the reflection point 54 are the ghost reflected echoes.

The reflection point 54 is positioned on the same line in the azimuth direction as the element 52d. Accordingly, as shown in FIG. 6B, in the transmission and the reception, the positions of the element 52e which is the center element and the reflection point 54 in the x direction are shifted in the x direction by one element, that is, by Le.

Since the coordinates of the element 52d whose position in the x direction conforms with the reflection point 54 are (x0, 0), the coordinates of the element 52e which is the center element become (x0+Le, 0), and the coordinates of the focus point 58 in the transmission become (x0+Le, df). Here, as described above, the coordinates of the reflection point 54 are (x0, z).

Accordingly, it is possible to calculate the length (the transmission path distance) Ltb of the transmission path 61 of the ultrasonic beam from the element 52e which is the center element to the reflection point 54 via the focus point 58 using Ltb=df+√{(z−df)²+Le²}. On the other hand, it is possible to calculate the length (the receiving path distance) Lrb of the receiving path 60 of the ghost reflected signal from the reflection point 54 to the element 52d directly above the reflection point 54 (the same position in the x direction (=the azimuth direction)), using Lrb=z.

Accordingly, a propagation distance Lub of ultrasonic waves in the case of ghost reflected echoes is Lub=Ltb+Lrb=df+√{(z−df)²+Le²}+z.

In this manner, a value obtained by dividing the propagation distance Lua of the ultrasonic waves which is the sum of the distance Lta of the transmission path 61 and the distance Lra of the receiving path 60 as determined by the geometric arrangement shown in FIG. 6A by the sound velocity is the propagation time of the true ultrasonic echoes. In addition, a value obtained by dividing the propagation distance Lub of the ultrasonic waves which is the sum of the distance Ltb of the transmission path 61 and the distance Lrb of the receiving path 60 as determined by the geometric arrangement shown in FIG. 6B by the sound velocity is the propagation time of the ghost reflected echoes.

The delay time is determined from the difference between the propagation time of the true ultrasonic echoes when the x coordinates of the reflection point 54 and the center element are the same and the propagation time of the ghost reflected echoes when the x coordinates of the reflection point 54 and the center element are shifted from each other by a single element interval.

The geometric models of FIG. 6A and FIG. 6B are each a model where the transmission path 61 goes via the focus point 58; however, the present invention is not limited thereto, and, for example, may be a path arriving at the reflection point 54 without going via the focus point 58.

In addition, the geometric models of FIG. 6A and FIG. 6B are each for the case of a linear probe; however, without being limited thereto, it is possible to perform the geometric calculation in the same manner from the shape of the probe even for other probes.

For example, in the case of a convex probe, it is possible to carry out the calculation in the same manner by setting the geometric model using the radius of the probe and angle of the element interval.

In addition, in the case of a steering transmission, it is possible to calculate the delay time of the true element data and the ghost element data in the vicinity of the true element data from the positional relationship between transmission elements and reflection points using a geometric model taking information on the transmission angle and the like into consideration.

Furthermore, without being limited to a method of calculating the delay time using a geometric model, by determining in advance the delay time for every measuring condition from the measuring results of measuring a high brightness reflection point in accordance with measuring conditions of the apparatus and storing the delay times in the apparatus, the delay time for the same measuring condition may be read out.

FIG. 6C shows the true element data 66 and the ghost element data 68.

In FIG. 6C, the data in the center in the azimuth direction is the true element data 66, that is, element data obtained by transmission and reception where the positions of the center element and the reflection point 54 in the x direction conform (element data where the element 52d is taken as the center element in the example in the diagram). In addition, pieces of data on both sides of the center are ghost element data, that is, element data obtained by transmission and reception where the positions of the center element and the reflection point 54 in the x direction do not conform (element data where the element 52c or the element 52e is taken as the center element in the example in the diagram).

In addition, FIG. 6D shows an example of the delay times of the pieces of ghost element data 68 with respect to the true element data 66 obtained by the geometric calculation described above. Centering on the true element data 66, pieces of the element data 68 of the ghost signals are delayed to be symmetrical in the x direction, that is, the azimuth direction, in terms of time.

In this manner, it is also possible to use the delay time calculated in the delay time calculator 48 of the element data processor 22 in the delay correction in the phasing addition section 38.

As will be described in detail below, in the present invention, by superimposing on element data, which is obtained by the transmission of an ultrasonic beam with a certain element of interest being the center element (the transmission and reception of the element of interest), another element data, which is obtained by the transmission of an ultrasonic beam with at least a part of the ultrasonic beam overlapping and with the center element being different, with the reception times of the ultrasonic echoes and the positions of the elements being matched, the processed element data (second element data) of the element of interest is generated (the element data of the element of interest is rebuilt).

In FIG. 6A, the reflection point 54 indicates the position of a certain sampling point (the output position of the element data) positioned directly below the element of interest (at the same position in the azimuth direction or on a straight line linking the element of interest and the focus point). In the present invention, the transmission and reception path to the sampling point in the transmission and reception of the element of interest is regarded as the transmission and reception path of the true element data and the transmission and reception path to the same sampling point in the transmission and reception of the ultrasonic waves where the center element is different (the transmission and reception from the adjacent elements) is regarded as the ghost transmission and reception path. The superimposition is performed by calculating the delay time from the difference between those transmission paths and matching times of pieces of element data using the delay time. In other words, the delay time is calculated and the superimposition of pieces of element data is performed assuming that element data obtained by the transmission and reception of the element of interest is the true element data and element data obtained by the transmission and reception where the center element is different is the ghost element data.

In the present invention, the superimposition of pieces of element data is performed by calculating the delay time with the same concept for all of the sampling points (the output positions of all the pieces of element data) and the processed element data of each of the elements is generated.

Here, in fact, even when a position of a sampling point (reflection point) is shifted in the azimuth direction (the x direction), the length of the receiving path (the receiving path distance Lrb) does not change. Accordingly, for each element of interest, the calculation of the delay time of a certain piece of element data from another piece of element data obtained through transmission and reception with a different center element may be performed for every sampling point along the depth direction (the y direction).

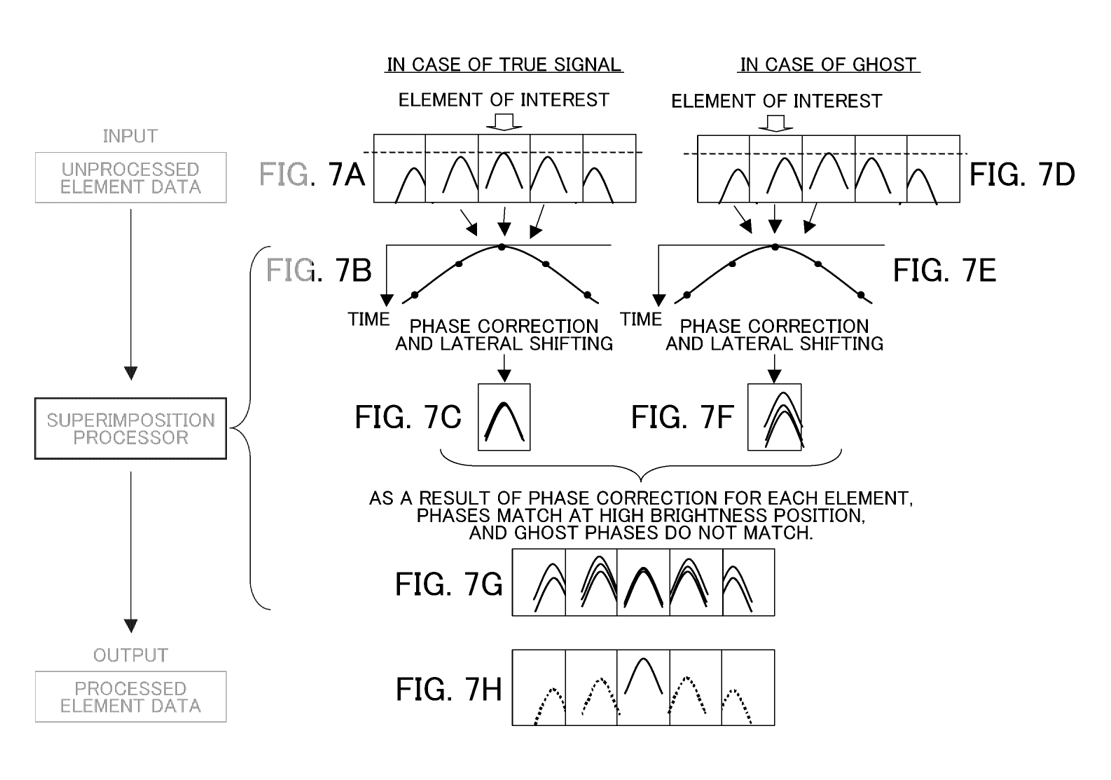

In addition, it is not necessary to know which piece of element data is the true element data in the superimposition process. That is, although described in detail with reference to FIGS. 7A to 7H below, in the superimposition process, the element data of the element of interest is automatically emphasized and remains when this element data is the true element data and the element data is cancelled when the element data is ghost element data. That is, in the case where the element data of the element of interest is the true element data, the process according to the delay time is matched and the signal is emphasized, whereas in the case where the element data of the element of interest is the ghost element data, the process according to the delay time does not match and the signal is cancelled.

Next, in the superimposition processor 49 of the element data processor 22 of the present invention, the superimposition process of pieces of element data is performed using the delay time calculated in the delay time calculator 48 in this manner.

Here, in the superimposition process in the superimposition processor 49, information on the superimposition processing method and the number of pieces of superimposition element data at the time of the superimposition is necessary, and this information may be input using the operating section 32 in advance, or may be stored in the storage 34 in advance.

FIGS. 7A to 7H show an example of the superimposition process performed in the superimposition processor 49. Here, the example shown in FIGS. 7A to 7H is the case where the number of pieces of element data is five and the number of pieces of superimposition element data is three.

FIG. 7A shows five pieces of element data lined up side by side obtained by carrying out the transmission and reception of the ultrasonic waves five times. In addition, FIG. 7A represents, for each piece of element data, the state where ultrasonic echoes are received after the ultrasonic beams are transmitted. The horizontal axis of each piece of element data represents a reception element, with the center element in the transmission and reception of the ultrasonic beam being positioned in the center in each piece of element data. The vertical axis represents the reception time. In this example, transmission and reception of the ultrasonic waves is performed five times by shifting the center element by one element every time, for example, from the element 52b to the element 52f.

FIGS. 7A to 7H show the state where one reflection point is present only directly below the center element in the middle element data. That is, out of the five pieces of element data, in the middle element data, the true ultrasonic echoes are received from the reflection point in the transmission and reception of the ultrasonic waves. That is, the element data in the middle is the true element data.

Regarding the four pieces of element data on both sides of the middle element data, the reflection point is not present directly below the center element in the transmission and reception of the ultrasonic waves. However, as the transmitted ultrasonic beam broadens, the ultrasonic beam hits the reflection point which is present directly below the transmission element of the middle element data, and element data of the resultant reflected echo generated thereby, that is, the ghost element data appears.

The further the ghost element data is positioned away from the true element data, the longer the propagation time of the ultrasonic waves up to the reflection point, and thus the reception time for the ghost element data is delayed compared to the true element data. In addition, the position of the reception element that first receives the ultrasonic echoes from the reflection point is shifted in the azimuth direction in this case.

Here, on the horizontal axis of each piece of element data in FIGS. 7A to 7H, the center element during the transmission of the ultrasonic beam is taken as the center. Accordingly, in the examples shown in FIGS. 7A to 7H, since transmission is carried out by shifting the center element by one element for each piece of the element data, the absolute positions of the elements in the azimuth direction in each piece of element data are shifted by one element. In other words, in the middle element data, the reception element which first receives the reflected signal from the reflection point is the center element, and in adjacent pieces of element data on both sides of the middle element data, the reception element is shifted by one element from that of the middle element data. That is, in the element data on the right side, the reception element is shifted by one element to the left, and in the element data on the left side, the reception element is shifted by one element to the right. Furthermore, in each piece of element data at either end, the reception element is shifted by two elements from that of the middle element data, that is, in the element data at the right end, the reception element is shifted by two elements to the left, and in the element data at the left end, the reception element is shifted by two elements to the right. In this manner, in addition to the presence of delay in the reception time compared to the true signal, in the ghost signal, the reception element is also shifted in terms of direction compared to the true signal.

FIG. 7B shows an example of the delay time of the reception time with respect to the element data in the middle of the five element data shown in FIG. 7A.

In the superimposition processor 49, in the case where the element data in the middle is set as the element data of the element of interest and the delay time shown in FIG. 7B is used, the delay time correction is performed on a certain number of pieces of element data to be superimposed (three pieces of element data in the example in the diagram) with the element data of the element of interest being centered; and pieces of unprocessed element data of three pieces of element data are superimposed after each piece of element data is shifted according to the difference in element position with respect to the element of interest (difference in position of the center element), i.e., shifted by one element in the azimuth direction toward either end in the example in the diagram, that is, with matched phases, and the resultant is determined as one superimposition-processed element data associated with the element data of the element of interest.

That is, in the present example, the processed element data of the element data of the element of interest is generated by superimposing the element data obtained by transmission and reception of the ultrasonic waves where the element adjacent to the element of interest is the center element (hereinafter, also referred to as the element data of the adjacent element) on the element data obtained by the transmission and reception of the ultrasonic waves where the element of interest is the center element (hereinafter, also referred to as element data of the element of interest).

The superimposition-processed element data of the element data of the element of interest obtained in this manner is shown in FIG. 7C.

As described above, the element data of the element of interest shown in FIG. 7A is true element data in which the reflection point is present directly below the center element (that is, the element of interest). In addition, the element data obtained by the transmission and reception where an element adjacent to the element of interest is the center element is also data of ultrasonic echoes where the ultrasonic waves reach the reflection point and reflected.

Accordingly, when the phase matching is performed by carrying out delay time correction and azimuth direction shifting on pieces of element data of the elements adjacent to, i.e., on both sides of the element of interest, the pieces of element data of the adjacent elements and the element data of the element of interest overlap at a high brightness position since their phases match as shown in FIG. 7C. Therefore, for example, when these pieces of element data are added, the element data value becomes a large value (high brightness value) and, for instance, when an average value is determined by averaging, the element data also becomes an emphasized value (high brightness value).

In contrast, FIG. 7D shows an example of a case with the same element data as FIG. 7A; however, the element data to the immediate left of the middle element data is the element data of the element of interest. That is, this example shows a case of the transmission and reception of ultrasonic waves where an element that is not present directly above the reflection point is the center element, and the center element is the element of interest. Accordingly, the element data where this element is the center element is ghost element data.

FIG. 7E is the same as FIG. 7B and shows an example of the delay time of the reception time with respect to the element data of the element of interest of the five pieces of element data shown in FIG. 7A. That is, since FIG. 7A and FIG. 7D are of the same element data, the delay time of the reception time with respect to the element data of the element of interest of the five pieces of element data shown in FIG. 7D is also the same.

In the superimposition processor 49, the delay time correction is performed for certain pieces of element data to be superimposed (three pieces of element data in the example in the diagram) with the element data of the element of interest being centered with the use of the delay time shown in FIG. 7E (that is, the same as FIG. 7B); and pieces of unprocessed element data of three elements are superimposed after each piece of element data is shifted according to the difference in element position with respect to the element of interest (difference in position of the center element), i.e., shifted by one element in the azimuth direction toward either end in the example in the diagram, and the resultant is determined as one superimposition-processed element data associated with the element data of the element of interest.

The superimposition-processed element data of the element data of the element of interest obtained in this manner is shown in FIG. 7F.

The element data of the element of interest shown in FIG. 7D is ghost element data. Therefore, even when phase matching is performed by performing delay time correction and azimuth direction shifting on pieces of unprocessed element data of the adjacent pieces of element data on both sides of the element data of the element of interest, as shown in FIG. 7F, the pieces of element data of the adjacent pieces of element data and the element data of the element of interest do not overlap because their phases do not match with each other. For this reason, since the phases do not match even when, for example, three pieces of element data are added, signals or the like where the phases are inverted cancel out each other, and thus the added value does not become large and, for example, a small value is obtained when the average value is determined by averaging.

For the other pieces of element data, as a result of performing the same delay time correction and azimuth direction shifting as those performed on the element data of the element of interest, FIG. 7G shows an overlapping state of three adjacent pieces of element data for each of five pieces of element data in the example in the diagram. With respect to these, FIG. 7H shows the results after, for example, an addition process or an averaging process is carried out as the superimposition process.

As shown in FIG. 7H, in the case of element data where a center element directly below which the reflection point is present shown in FIG. 7A is the element of interest as shown in FIG. 7A, the element data of the true signal is determined as superimposition-processed element data having a high brightness value. In contrast, in all of the four pieces of element data (the two pieces of element data on either side of the middle element data), for the ghost element data, the element data with their phases not matching with each other are added or averaged. Therefore, since the element data cancel out each other, the value of the ghost superimposition-processed element data is lower than that of the superimposition-processed element data having a high brightness value which is element data of a true signal, and it is possible to reduce the influence of the ghost element data on the true element data, or it is possible to reduce the influence thereof to the ignorable level.

That is, one or more pieces of element data which are obtained by transmission and reception of ultrasonic waves where the transmission regions of the ultrasonic beams overlap and where the center elements are different are superimposed on element data (element data of the element of interest) where a certain element is set as the element of interest and which is obtained by transmission of an ultrasonic beam with the element of interest being the center element, and thus processed element data corresponding to the element data of the element of interest is generated. In other words, the element data of the element of interest is rebuilt (corrected) using the element data obtained through transmission and reception where the center element is different. Due to this processing, the brightness level of the true element data can be increased and it is possible to decrease the ghost element data.

Therefore, as will be described below, according to the present invention which performs determination of the sound velocity using the processed element data, it is possible to determine the sound velocity in the subject with high precision even with one focus point without influence of the ghost by using element data equivalent to that obtained by linking focus points at many points on a sound ray transmitted, that is, element data obtained by the transmission of the ultrasonic waves with multiple virtual focus points (the reception data (ultrasound image data)).

In addition, similarly, since it is possible to generate the ultrasound image with element data without influence of the ghost, that is, element data equivalent to that obtained by linking focus points at all points on a sound ray, by performing phasing addition and a detection process on the processed element data, generating the reception data, and generating the ultrasound image, it is possible to generate an ultrasound image with high image quality, high brightness, and excellent sharpness.

The generation of the processed element data is also referred to as a multi-line process in the following description.

As described above, the processed element data generated in the multi-line process is element data without influence of the ghost and equivalent to that obtained by linking focus points at many points on a sound ray transmitted, that is, element data obtained by the transmission of the ultrasonic waves with multiple virtual focus points.

Therefore, according to the present invention which performs the determination of the sound rays using the processed element data, even with the transmission of ultrasonic waves with one focus point on one sound ray, it is possible to determine the sound velocity with high precision equal to or higher than that in the case where the transmission of the ultrasonic waves is performed with many focus points on one sound ray. In addition, since the sound velocity can be determined with high precision by the transmission of ultrasonic waves with one focus point on one sound ray, it is also possible to prevent a decrease in the frame rate which accompanies the determination of the sound velocity (updating of the sound velocity). For this reason, the present invention is effective in the motion picture photographing mode.

In the multi-line process above, the processed element data of the element data of the element of interest is generated by superimposing the pieces of element data where the center elements are different and which are obtained by the transmission of a plurality of ultrasonic beams whose transmission directions are parallel (the angles are the same); however, the present invention is not limited thereto.

For example, the processed element data may be generated by superimposing the pieces of element data where the center elements are the same and which are obtained by the transmission of a plurality of ultrasonic beams whose transmission directions (angles) are different. At this time, among transmitted ultrasonic beams, an ultrasonic beam for use in generating the processed element data of the element data (that is, a direction of the sound ray for use in generating the processed element data) may be set by default according to the examination site, the type of probe, or the like, or may be selected by the operator.

The processed element data may be generated using both of the element data where the center elements are different and which are obtained by the transmission of parallel ultrasonic beams and the element data where the center elements are the same and which are obtained by the transmission of ultrasonic beams in different transmission directions.

In the present invention, the center element is the element in the center in the azimuth direction in the case where the number of transmission openings (the number of elements which perform the transmission of the ultrasonic waves) is an odd number, and the center element is any of the elements in the center in the azimuth direction or is set to a virtual element which is assumed to be present in the middle between elements in the center in the case where the number of openings is an even number in the azimuth direction In other words, calculation is performed assuming that there is a focus point on a line in the middle of the opening in the case where the number of openings is an even number.

As the superimposition processing method in the superimposition processor 49, an average value or a median value may be taken instead of only adding, or addition may be carried out after multiplication with a coefficient. Here, taking the average value or the median value may be considered equivalent to applying an averaging filter or a median filter in the element data level; however, an inverse filter or the like used in a normal image processing may also be applied instead of the averaging filter and the median filter. Alternatively, the invention is not limited thereto and the superimposition process may be changed based on the feature amount of each piece of element data to be superimposed, for instance, the pieces of element data to be superimposed are compared and when they are similar, the maximum value is taken; when they are not similar, the average value is taken; and when there is bias in the distribution, the intermediate value is taken.

In addition, the number of pieces of element data to be superimposed on the element data of the element of interest is not limited to two in the example in the diagram and may be one or may be three or more. That is, the number of pieces of element data to be superimposed on the element data of the element of interest may be appropriately set according to the required processing speed (the frame rate or the like), image quality, or the like.

Here, it is desirable that the number of pieces of element data to be superimposed on the element data of the element of interest accord with the degree of the spread of the beam width of the ultrasonic beam. Accordingly, in the case where the beam width changes according to the depth, the number of pieces of element data to be superimposed may also be changed according to the depth.

In addition, since the beam width depends on the number of transmission openings, the number of pieces of element data to be superimposed may be changed according to the number of the transmission openings. Alternatively, the number of pieces of element data to be superimposed may be changed based on the feature amount of the image such as the brightness value or the like, or the optimum number of pieces of element data to be superimposed may be selected based on images generated by changing the number of pieces of element data to be superimposed among a plurality of patterns.

As described above, the element data processor 22 outputs the generated processed element data to the image generator 24 (the phasing addition section 38) and the sound velocity determiner 23.

In the image generator 24 to which the processed element data is supplied, as described above, the phasing addition section 38 carries out phasing addition on the processed element data to perform a reception focusing process to thereby generate the reception data, and the detection processor 40 carries out attenuation correction and an envelope detection process on the reception data to thereby generate B-mode image data.

In addition, in the image generator 24, the DSC 42 raster converts the B-mode image data into image data corresponding to a normal television signal scanning method, and the image processor 44 carries out a predetermined process such as a gradation process.

The image processor 44 stores the generated B-mode image data in the image memory 46 and/or sends the generated B-mode image data to the display controller 26 to display a B-mode image of the subject on the monitor 28.

On the other hand, the sound velocity determiner 23 determines the sound velocity (calculates the sound velocity) of the ultrasonic waves in the subject using the supplied processed element data.

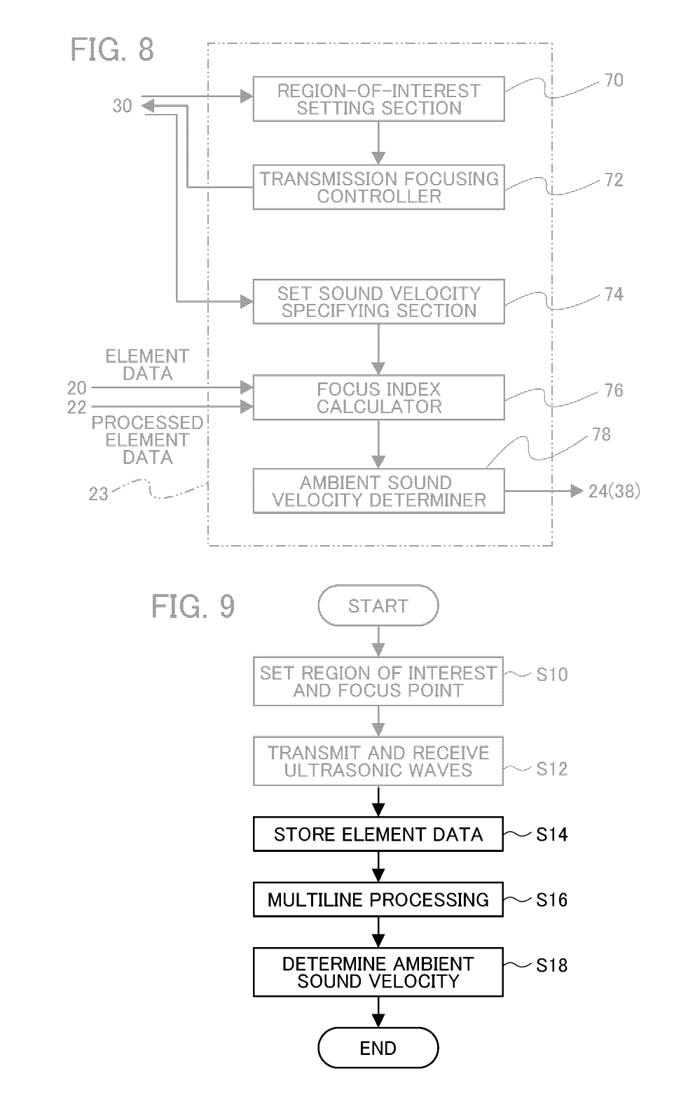

FIG. 8 is a block diagram conceptually showing the configuration of the sound velocity determiner 23.

As shown in FIG. 8, the sound velocity determiner 23 has a region-of-interest setting section 70, a transmission focusing controller 72, a set sound velocity specifying section 74, a focus index calculator 76, and an ambient sound velocity determiner 78.

The region-of-interest setting section 70 sets a region of interest in the B-mode image (in the ultrasound image) according to instructions from the controller 30.

In the sound velocity determiner 23, the sound velocity of the subject is determined for every region of interest.

In the present embodiment, the region-of-interest setting section 70 divides the entire screen of the B-mode image into a grid pattern and sets each of the resulting segments as a region of interest.

The number of divisions (the number of the segments) may be set in advance by default, or the operator may set any number in the azimuth direction and/or the depth direction. In the case where the number of the divisions is set by default, a set value may vary depending on the image size or the site to be observed. Furthermore, it may be possible for the operator to select one from a plurality of choices of the number of divisions set in advance.

In the present invention, the region of interest is not limited to the regions in the grid pattern obtained by dividing the B-mode image.

For example, all of the pixels (the positions (regions) corresponding to all of the pixels) generating the reception data (B-mode image data) may be set as regions of interest. In other words, in the embodiment where the screen is divided as described above, the screen may be divided into a grid pattern corresponding to all of the pixels generating the reception data. In addition, the entire screen may be set as one region of interest.

Alternatively, instead of the entire screen, a part of the screen which is set in advance or selected from a plurality of choices may be divided into a grid pattern, and the segments thereof may be individually set as regions of interest. In addition, instead of the entire screen, the region of interest may be set in correspondence with a region of interest (ROI) set by the operator. Here, in the case where the region of interest is set in a part of the screen or in the ROI, the division may be performed in the same manner as for the entire screen. In addition, the operator may select the setting of the region of interest in the entire screen or the setting of the region of interest in the ROI.

In addition, the form of the division is not limited to a grid pattern. For example, in the case of a B-mode image with a fan shape such as an ultrasound image using a convex probe, the form of the division may also be set to a fan shape according to this. Also in such a case, it is possible to use each embodiment described above.

In cases where an image is greatly changed or where an observation condition such as observation magnification or observation depth is modified, or in other cases, a region of interest may be changed or updated, and such change or update of a region of interest may be carried out in response to an instruction by the operator. The case where an image is greatly changed described above refers to the case where, for example, a change value in a feature amount of the image exceeds a threshold value.

The region-of-interest setting section 70 also sets a focus point (the position of the focus point) in order to transmit the ultrasonic waves (perform transmission focusing) corresponding to the determination of the sound velocity for the set region of interest.

The focus point may be set by default in advance according to the observation site, the number of sound rays, the number of transmission and reception openings, the type of the probe 12, or the like, or the operator may select or input instructions. One among the default setting, and the operator's instruction, and the like may be selected for use.

As described above, the present invention, which determines the sound velocity using the processed element resulting from the superimposition of pieces of element data, can perform the transmission using multiple virtual focus points. Accordingly, in the motion picture photographing mode, basically, the focus point is set to one position for one sound ray. With this, it is possible to determine the sound velocity even during taking a moving image.

The position of the focus point in the motion picture photographing mode is desirably set at the deepest position on the measuring screen or at a still deeper position. With this, since a spreading transmission beam is transmitted on the display screen, when the superimposition process is performed by the multi-line process, the actual signal is enhanced and the ghost signal is suppressed by the superimposition of the large number of pieces of element data, whereby element data can be obtained by pseudo focusing regardless of the depth. However, in the case where the precision of the superimposition is decreased due to the influence of non-uniformity in a living body, or the like, since the quality of the signal becomes lower than that with the actual focus point, the performance could be inferior to the actual focus point. For this reason, in the case where the frame rate is not related, for instance in the case of a still image, it is desirable to carry out the measurement with a conventional method.

In addition, in the calculation of the sound velocity value in the moving image, since the element data is data in which focusing is established at any depth owing to the multi-line process, it is possible to freely set the setting intervals of the ROI. For example, it is possible to obtain a sound velocity value with improved spatial resolution by setting the ROI intervals more finely than in a still image.

The transmission focusing controller 72 sends a transmission focusing instruction to the controller 30 so that the transmission section 14 performs the transmission focusing according to the region of interest and the focus point set by the region-of-interest setting section 70.

The set sound velocity specifying section 74 specifies a set sound velocity in order to perform reception focusing with respect to the reception data under the control of the controller 30 in the determination of the ambient sound velocity.

The focus index calculator 76 calculates the focus index of the reception data by performing reception focusing with respect to the reception data for each of a plurality of set sound velocities specified by the set sound velocity specifying section 74 using the element data in the element data storage 20 or the processed element data generated by the element data processor 22.

The ambient sound velocity determiner 78 determines the ambient sound velocity of the region of interest based on the focus index for each of the plurality of set sound velocities.

Below, detailed description will be given of a method for determining the sound velocity in the ultrasound diagnostic apparatus 10 with reference to the flow chart shown in FIG. 9 with taking a method for determining a sound velocity in the motion picture photographing mode as an example.

In the ultrasound diagnostic apparatus 10, when determining the ambient sound velocity, first, the region-of-interest setting section 70 sets the region of interest and the focus point according to instructions from the controller 30 as described above (step S10).

Here, in the present invention, the timing at which the ambient sound velocity is determined (the update timing of the ambient sound velocity) is not particularly limited and may be the same as a known ultrasound diagnostic apparatus. For example, the determination of the ambient sound velocity may be performed only one time according to the instruction of the measurement start instructions, may be performed when the image is greatly changed (when a change value of a feature amount of the image exceeds a threshold, or the like), may be performed every predetermined number of frames or every time a predetermined time passes as determined as appropriate, or may be performed according to the input instructions of the operator. Two or more timings for the sound velocity determination as described above may be appropriately selected.

Regardless of the timing at which the ambient sound velocity is determined, in the motion picture photographing mode in which the multi-line process is performed, since the transmission is performed with one focus point for one sound ray, it is possible to determine the ambient sound velocity even in the motion picture photographing mode.

According to the setting of the region of interest, the transmission focusing controller 72 sends a transmission focusing control instruction to the controller 30 so that the transmission section 14 executes the transmission focusing with respect to the set region of interest and focus point.

In response thereto, the transmission section 14 transmits the ultrasonic beam to the subject by driving the probe 12 (corresponding ultrasound transducers (elements) in the transducer array 36), the ultrasonic echoes reflected by the subject are received by the elements, and analog reception signals are output from the ultrasound transducers (elements) to the receiving section 16 (step S12).

The receiving section 16 carries out a predetermined process such as amplification on the analog reception signals and supplies them to the A/D converter 18.

The A/D converter 18 A/D converts the analog reception signals supplied from the receiving section 16 to alter the signals into element data which are digital reception signals.

The element data is stored in the element data storage 20 (step S14).

When the element data is stored in the element data storage, the element data processor 22 generates the processed element data by performing the multi-line process described above.

That is, as shown in FIGS. 7A to 7H, for the element of interest and adjacent elements on both sides thereof, the element data processor 22 calculates the delay times of the pieces of element data of the adjacent elements with respect to the element data of the element of interest, performs delay time correction and azimuth direction shifting on the pieces of element data of the adjacent elements, and generates the processed element data of the element of interest by superimposing the pieces of element data of the adjacent elements on both sides on the element data of the element of interest (step S16).

The element data processor 22 supplies the generated processed element data to the sound velocity determiner 23 (the focus index calculator 76). Here, the element data processor 22 also supplies the generated processed element data to the image generator 24, and the image generator 24 generates the ultrasound image (B-mode image data) using the processed element data, as described above.

The sound velocity determiner 23 determines the sound velocity of the ultrasonic waves in the subject using the supplied processed element data (step S18).

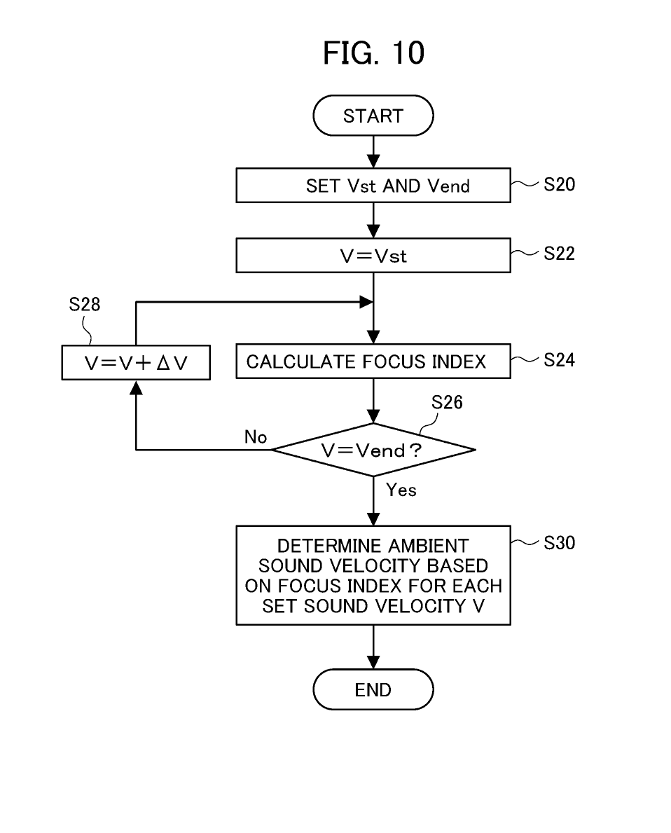

FIG. 10 shows a flow chart of an example of the sound velocity determining method in the sound velocity determiner 23. Here, in the present invention, the sound velocity determining method in the sound velocity determiner 23 is not limited to this method and it is possible to use various sound velocity determining methods (methods of calculating the sound velocity) performed in ultrasound diagnostic apparatuses.

When the processed element data is supplied, the sound velocity determiner 23 stores the processed element data in a predetermined site as necessary and, first, sets a start sound velocity Vst and an end sound velocity Vend of the set sound velocity V (step S20), and then sets the start sound velocity Vst to the set sound velocity V (step S22).

Set sound velocities including the start sound velocity Vst and the end sound velocity Vend may be set in advance as default values. Alternatively, only the start sound velocity Vst and the end sound velocity Vend may be input by the operator as desired, while only the incrementing step therebetween (predetermined step sound velocity amount ΔV) may be set as a default value. As a further alternative, the operator may input the start sound velocity Vst, the end sound velocity Vend and the incrementing step as desired. In addition, in the case where the set sound velocity or the incrementing step of the set sound velocity is set by default, a plurality of types of set sound velocities are set according to the observation site, the sex of the subject, or the like, and appropriate one can be selected by the operator.

In the present example, as an example, 1410 m/sec is set as the start sound velocity Vst and 1570 m/sec is set as the end sound velocity Vend and, accordingly, the set sound velocity is set at intervals of 40 m/sec as the predetermined incrementing step.

Next, the focus index calculator 76 calculates the focus index of the reception data by carrying out reception focusing with respect to the processed element data for each of the plurality of set sound velocities specified by the set sound velocity specifying section 74 for each of the regions of interest (step S24).

Specifically, the focus index calculator 76 calculates, as the focus index, an integrated value, a squared integral value, a peak value, a degree of sharpness (sharpness), a contrast, a brightness value, a half-width, a frequency spectrum integration, a frequency spectrum integral value or squared integral value normalized by a DC component or a maximum value, an autocorrelation value, and the like of the reception data (the ultrasound image data/ultrasound image) in the region of interest.

Next, the sound velocity determiner 23 determines whether or not the set sound velocity V has reached the end sound velocity Vend in the set sound velocity specifying section 74 (step S26), and, if the set sound velocity V is less than the end sound velocity Vend (No), the predetermined step sound velocity amount ΔV, that is, 40 m/sec in the present example, is added to the set sound velocity V (step S28) to calculate the focus index of the region of interest.

This routine is repeated and when it is determined that the set sound velocity V has reached the end sound velocity Vend (Yes), the ambient sound velocity of the region of interest is determined by the ambient sound velocity determiner 78 based on the focus index for each of the plurality of set sound velocities by, for example, setting the set sound velocity with the highest focus index to the ambient sound velocity of the region of interest (step S30). For example, by setting the brightness of the ultrasound image as the focus index, the sound velocity with which the ultrasound image having the highest brightness is obtained in the region of interest is set as the ambient sound velocity of the region of interest.

That is, the ambient sound velocity in the present example is the average sound velocity of a region between the ultrasound probe 12 (the transducer array 36 (ultrasound transducers)) and a certain region of interest when the sound velocity from the probe 12 to the region of interest is assumed to be constant.

As described above, the sound velocity determiner 23 performs the determination of the ambient sound velocity in this manner in all of the set regions of interest. The ambient sound velocity determined by the sound velocity determiner 23 is stored in the element data storage 20 in association with positional information in the ultrasound image.

In addition, the determined ambient sound velocity is supplied to the phasing addition section 38 and used in the reception focusing process. With this, an ultrasound image based on the ambient sound velocity is displayed on the monitor 28.

In the determination of the ambient sound velocity, even in the case where A/D converted element data is used instead of the processed element data generated by the multi-line process, it is possible to determine the ambient sound velocity as described above in the same manner as with the processed element data generated by the multi-line process. Accordingly, detailed description of the method for determining the ambient sound velocity using the element data will be omitted. Also in this case, the ambient sound velocity determined by the sound velocity determiner 23 is stored in the element data storage 20 in association with the positional information in the ultrasound image.

In addition, the ambient sound velocity value determined using the element data is supplied to the phasing addition section 38 and used in the reception focusing process. An ultrasound image based on the ambient sound velocity is displayed on the monitor 28.

The ultrasound diagnostic apparatus 10 basically has the above configuration.

The ultrasound diagnostic apparatus 10 has the motion picture photographing mode and the still picture photographing mode as described above.

In the motion picture photographing mode, a moving image is taken by continuously generating the ultrasonic beams in time series as described above. At this time, transmission and reception is performed with single focus to obtain element data, the multi-line process described above is performed based on the element data, and processed element data is obtained. The phasing addition process is carried out on the processed element data, B-mode image data is obtained, and an ultrasound image is displayed on the monitor 28 as a moving image. For example, when obtaining the element data, the element data is obtained while shifting the element assuming the center in the arrangement direction of the elements, that is, while scanning in the arrangement direction.

Here, in the motion picture photographing mode, it is not always necessary to use the single focus. At least with a frame rate which is able to be used for a moving image, for example, with a frame rate of 5 fr/sec or more, focus points may be plural.

On the other hand, in the still picture photographing mode, as described above, the transmission and reception is performed with multi-focus in the same manner as the techniques of the related art and the element data is obtained. The phasing addition process is carried out on the element data, data of one line of an ultrasound image is generated based on one piece of element data, thereafter B-mode image data is obtained and the ultrasound image is displayed on the monitor 28 as a still image. Also in this case, for example, the element data is obtained while shifting the element assuming the center in the arrangement direction of the elements, that is, while scanning in the arrangement direction.

Here, the scanning direction and the scanning method for obtaining the ultrasound image for both of the motion picture photographing mode and the still picture photographing mode are not particularly limited and it is possible to appropriately use a known method or system.

In addition, in the case where the mode is switched from the motion picture photographing mode to the still picture photographing mode, for a focus point which corresponds to a focus point of a moving image out of a large number of focus points used in generating a still image, it is also possible to use data obtained in the motion picture photographing mode. With this, it is possible to shorten the time necessary to generate the still image.

In the still picture photographing mode, in comparison with the motion picture photographing mode, the frame rate need not be considered, and a plurality of focus points are set for one sound ray (one line of an ultrasound image), so that the image quality of the ultrasound image is better than that of the motion picture photographing mode. The position of the focus points may be the same for all of the sound rays, or sound rays with different focus points may be mixed.

In addition, in the still picture photographing mode, it is possible to obtain an ambient sound velocity value with improved spatial resolution in comparison with the motion picture photographing mode since the multi-focus is used.

In addition, the still picture photographing mode may also have a configuration where the transmission and reception are performed with single focus in the same manner as the motion picture photographing mode to obtain (first) element data, the multi-line process described above is performed to determine processed element data, and image data is generated from the processed element data. At that time, by switching the photographing mode, it is possible to change the measuring conditions such as the number of focus points and the focus point positions (the conditions of the transmission and reception of the ultrasonic waves) and the processing conditions of the multi-line process such as the number of pieces of element data to be superimposed in the multi-line process. For example, in the case where the mode is switched from the still picture photographing mode to the motion picture photographing mode by switching the photographing mode, by reducing the number of pieces of element data to be superimposed in the multi-line process, it is possible to ensure the moving image performance by reducing the load of the data processing in the motion picture photographing mode.

Next, description will be given of a method of photographing an ultrasound image using the ultrasound diagnostic apparatus 10.

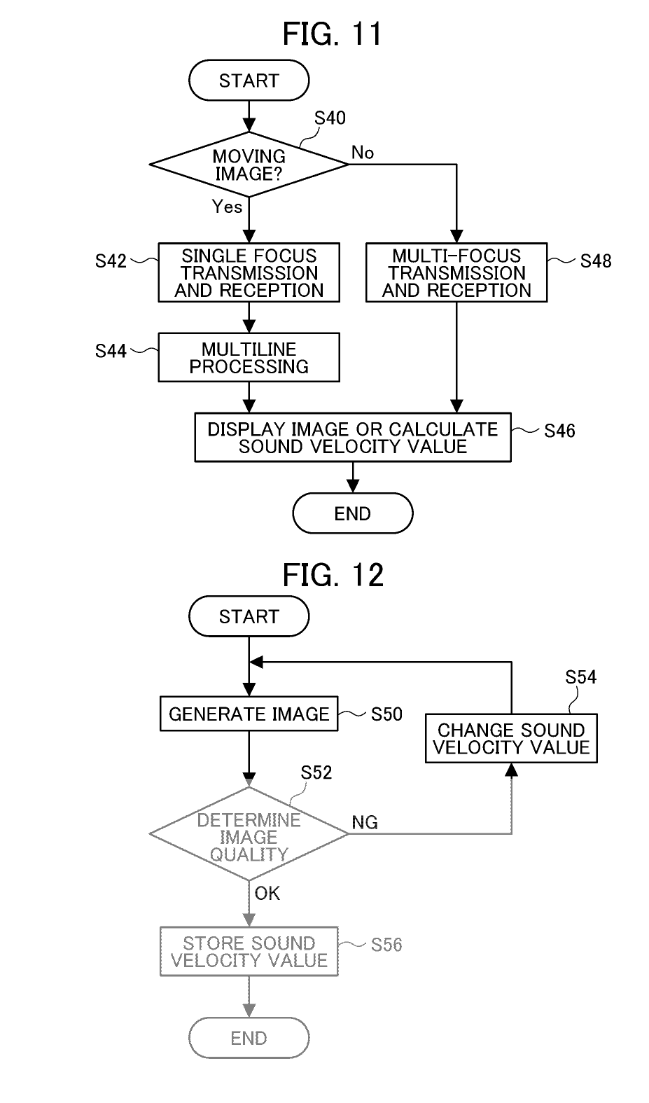

FIG. 11 is a flow chart for describing the still picture photographing mode and the motion picture photographing mode of the first ultrasound diagnostic apparatus of the embodiments of the present invention.

In the ultrasound diagnostic apparatus 10, as shown in FIG. 11, it is determined whether or not the mode is the motion picture photographing mode (step S40). Whether or not the mode is the motion picture photographing mode is determined based on the operation of the freeze button.

In the case of the motion picture photographing mode where the operation of the freeze button is released, transmission and reception is performed with single focus (step S42). Then, the multi-line process is performed based on the received element data (step S44). Then, based on the processed element data, the moving image of the ultrasound image is displayed, or the sound velocity value is calculated (step S46).

On the other hand, in step S40, in the case where the mode is not the motion picture photographing mode, that is, in the case of the still picture photographing mode where the freeze button is operated, the transmission and reception is performed with multi-focus (step S48). Then, the phasing addition process and the like are carried out on the received element data and a still image of the ultrasound image is displayed, or the sound velocity value is calculated (step S46).

In this manner, a picture of a subject in the motion picture photographing mode can be taken although its image quality is worse than in the related art, and a picture of sites to be precisely observed, or the like, can be taken in the still picture photographing mode with an image quality as in the related art. In addition, conventionally, it was possible to calculate the sound velocity value (the ambient sound velocity value) only in the still picture photographing mode; however, it is possible to calculate the sound velocity value (the ambient sound velocity value) even in the motion picture photographing mode.

Here, the computer readable recording medium having stored therein the program of the present invention is for causing a computer in the ultrasound diagnostic apparatus 10 to execute various types of photographing methods in the motion picture photographing mode and the still picture photographing mode shown in FIG. 11 described above. In addition, the computer readable recording medium having stored therein the program of the present invention causes each of the sections of the ultrasound diagnostic apparatus 10 to perform the various types of processes described above.

In addition, the generation of the ultrasound image and the determination of the sound velocity may be performed at the same time or may be performed separately. That is, the generation of the ultrasound image as well as the determination of the sound velocity may be performed using the element data obtained by the transmission and reception of one set of ultrasonic waves for one frame, or the generation of the ultrasound image and the determination of the sound velocity may be separately performed using different element data obtained through a different sequence of transmission and reception. The determination of the sound velocity may be performed for each frame, or may be performed once per a number of frames.

In the ultrasound diagnostic apparatus 10, the multi-line process is described with an example of using A/D converted element data; however, it is also possible to carry out the multi-line process using the reception data after phasing addition. In this case, a line which is a reference for the phasing addition is matched in each piece of element data (the line which is a reference for the phasing addition is shifted from the center line of each piece of element data), phasing addition is performed on each piece of element data to generate reception data, and the multi-line process described above is performed using the reception data.

Alternatively, after performing only lateral shifting (refer to FIGS. 7A to 7H and the like) with respect to each of the first element data, phasing addition is carried out to generate reception data, and the multi-line process may be performed using the reception data.

At the time of the multi-line process using the reception data after phasing addition, the ambient sound velocity is, for example, determined as shown in FIG. 12. At this time, the sound velocity determiner 23 has a function of carrying out the ambient sound velocity determining process described below.

First, image generation is carried out using the element data after the multi-line process is performed on the reception data after phasing addition (hereinafter, referred to as the element data after processing) (step S50). The image generation generates B-mode image data by carrying out correction of attenuation according to the depth and envelope detection processing on the element data after processing in the same manner as the detection processor 40.

Then, the image quality of the generated image is determined (step S52). In step S52, when the image quality is determined to be not good (NG), the sound velocity value is changed within a search range (step S54), the phasing addition process, the multi-line process, and the image generation are performed, and the image quality is determined again. In step S52, while the sound velocity value is changed within a search range until the image quality is determined to be good (step S54), the determination of the image quality (step S52) is repeated to find the optimum sound velocity value.

In step S52, when the image quality is determined to be good, the sound velocity value is stored as the ambient sound velocity value (step S56). The ambient sound velocity value determined in this manner can be used in the phasing addition process. In addition, the ambient sound velocity value is stored in association with the positional information of the ultrasound image in the element data storage 20.

For the determination of the image quality, for example, the sharpness value of the image data of the generated image is used. In addition, it is also possible to use the values given as the focus indexes in the foregoing explanation on the focus index calculator 76. The search range of the sound velocity value can be set in the same manner as the method for setting the set sound velocity in the set sound velocity specifying section 74 of the sound velocity determiner 23 described above.

Next, description will be given of a second embodiment of the present invention.

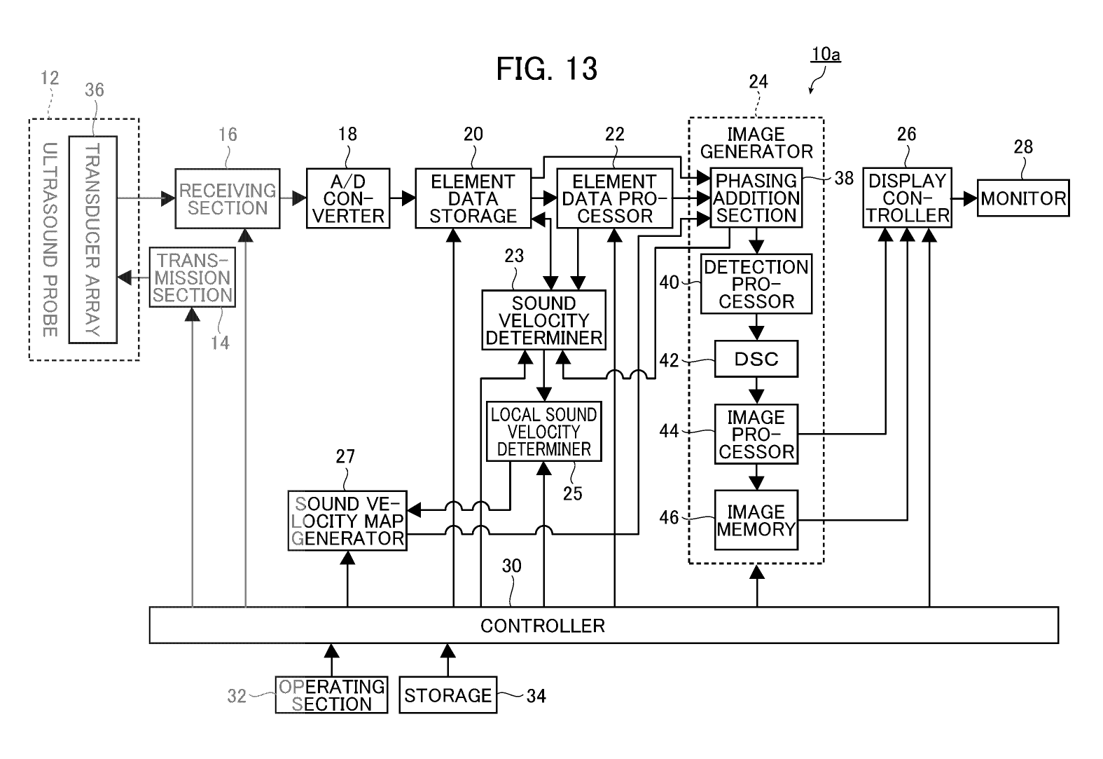

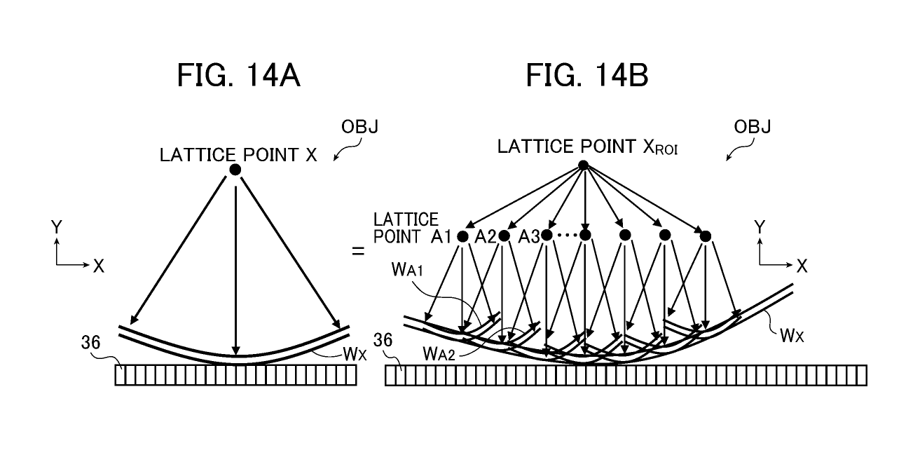

FIG. 13 is a block diagram illustrating an ultrasound diagnostic apparatus of the second embodiment of the present invention. FIGS. 14A and 14B are schematic diagrams for describing a calculation process of a local sound velocity value.

An ultrasound diagnostic apparatus 10a illustrated in FIG. 13 is different from the ultrasound diagnostic apparatus 10 illustrated in FIG. 1 in that a local sound velocity determiner 25 and a sound velocity map generator 27 are provided. Since the configuration is the same as that of the ultrasound diagnostic apparatus 10 illustrated in FIG. 1 in other respects, detailed description thereof will be omitted.

The local sound velocity determiner 25 is connected with the sound velocity determiner 23, and the sound velocity map generator 27 is connected with the local sound velocity determiner 25. The local sound velocity value determined by the local sound velocity determiner 25 is output to the sound velocity map generator 27 and the phasing addition section 38. The local sound velocity determiner 25 and the sound velocity map generator 27 are connected with the controller 30 and controlled by the controller 30.

The ultrasound diagnostic apparatus 10a can calculate a local sound velocity value and generate a sound velocity map based on the local sound velocity. Here, the local sound velocity is a sound velocity in an arbitrary site in the subject.

The local sound velocity determiner 25 determines the local sound velocity using the ambient sound velocity value. Below, description will be given of the calculation process of the local sound velocity value.

FIGS. 14A and 14B are diagrams schematically showing the calculation process of the local sound velocity value.

For the determination of the local sound velocity value, for example, it is possible to use the method disclosed in JP 2010-99452 A filed by the applicant of the present application.

In this method, paying attention to received waves Wx reaching a transducer array 36 from a lattice point X which is a reflection point in the subject during the transmission of ultrasonic beams into the subject as shown in FIG. 14A, a lattice point representing a region of interest ROI in the subject OBJ is set as X_{ROI}, and lattice points arranged at equal intervals in the XY direction at positions shallower than the lattice point X_{ROI} (that is, closer to the transducer array 36) are set as A1, A2, . . . as shown in FIG. 14B, and the sound velocities at least between the lattice point X_{ROI} and the respective lattice points A1, A2, . . . are assumed to be constant.

In the present example, (T and a delay time Δt) of reception waves (W_{A1}, W_{A2}, . . . ) from the lattice points A1, A2, . . . are assumed to be known, and the local sound velocity value at the lattice point X_{ROI} is determined from the positional relationship between the lattice point X_{ROI} and the lattice points A1, A2, . . . . Specifically, according to the Huygens' principle, the fact that a reception wave W_{X} from the lattice point X_{ROI} and a reception wave W_{SUM} determined by virtually synthesizing reception waves from the lattice points A1, A2, . . . are identical is used. The value of assumed sound velocity where the difference between the reception wave W_{X} and the virtual synthesized reception wave W_{SUM} is minimum is set as the local sound velocity value at the lattice point X_{ROI}.

Here, the range and number of the lattice points A1, A2, . . . , used in the calculation for determining the local sound velocity value at the lattice point X_{ROI} are determined in advance. Here, since the error in the local sound velocity value becomes large when the range of the lattice points used in the local sound velocity value calculation is wide and the error from a virtual reception wave becomes large when the range of the lattice points is narrow, the range of the lattice points is determined by finding a balance between the above factors.

The interval of the lattice points A1, A2, . . . in the X direction is determined based on a balance between the resolution and the processing time. The interval of the lattice points A1, A2, . . . in the X direction is from 1 mm to 1 cm as one example.

The error of the error calculation becomes large when the interval of the lattice points A1, A2, . . . in the Y direction is narrow and the error in the local sound velocity value becomes large when the interval is wide. The interval of the lattice points A1, A2, . . . in the Y direction is determined based on the setting of the image resolution of the ultrasound image. The interval of the lattice points A1, A2, . . . in the Y direction is 1 cm as one example.

In the case where the interval of the lattice points A1, A2, . . . is wide, the calculation of the synthesized wave is difficult, and therefore fine lattice points may be generated by interpolation.

The ambient sound velocity value of the entire region of interest is input to the local sound velocity determiner 25. In the local sound velocity determiner 25, a starting pixel of interest where the calculation of the local sound velocity value is started is set and the calculation of the local sound velocity value of the pixel of interest is performed.

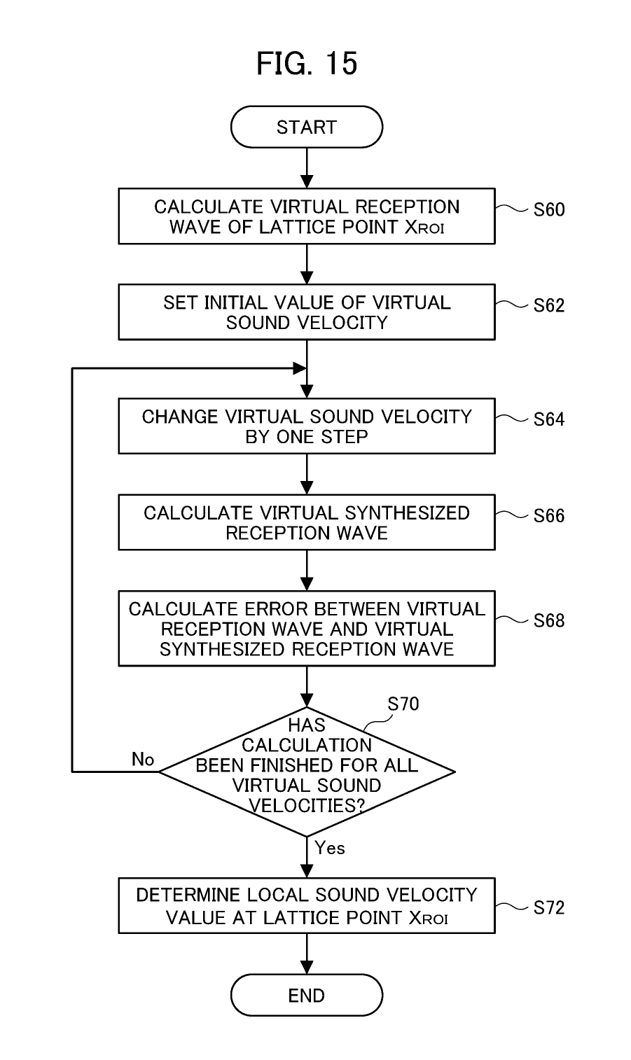

Below, description will be given of the method for determining the local sound velocity value of the pixel of interest using the flow chart shown in FIG. 15.

First, based on the ambient sound velocity value at the lattice point X_{ROI}, a waveform of the virtual reception wave W_{X} when the lattice point X_{ROI} is set as the reflection point is calculated (step S60).

Next, the initial value of the assumed sound velocity at the lattice point X_{ROI} is set (step S62). Then, the assumed sound velocity is changed by one step (step S64) and the virtual synthesized reception wave W_{SUM} is calculated (step S66). When the local sound velocity value at the lattice point X_{ROI} is assumed to be V, the times taken for the ultrasonic waves propagated from the lattice point X_{ROI} to reach the lattice points A1, A2, . . . are X_{ROI}A1/V, X_{ROI}A2/V, . . . . Here, X_{ROI}A1, X_{ROI}A2, . . . are distances between the respective lattice points A1, A2, . . . and the lattice point X_{ROI}. Since the ambient sound velocity values at the lattice points A1, A2, . . . has been determined by the sound velocity determiner 23 and are known, it is possible to determine the reception waves from the lattice points A1, A2, . . . in advance. Accordingly, by synthesizing the reflected waves (ultrasonic echoes) respectively emitted from the lattice points A1, A2, . . . with the delays X_{ROI} A1/V, X_{ROI}A2/V, . . . , it is possible to determine the virtual synthesized reception wave W_{SUM}.

Here, since the process described above is performed on the element data in practice, the times (T1, T2, . . . ) taken to reach the lattice points A1, A2, . . . from the lattice point X_{ROI} are represented by the following formula (3), where X_{A1}, X_{A2}, . . . are distances in the scanning direction (the X direction) between the respective lattice points A1, A2, . . . and the lattice point X, and Δt is the time interval of the lattice points in the Y direction.

[Formula 1]

T1=\sqrt{(X_{A1}/V)²+(Δt/2)²},

T2=\sqrt{(X_{A2}/V)²+(Δt/2)²},

T3= . . . (3)

It is possible to obtain the virtual synthesized reception wave W_{SUM} by synthesizing the reception waves from the lattice points A1, A2, . . . using delays obtained by adding the time (Δt/2) taken to reach the lattice point X_{ROI} from the lattice point An associated with the same sound ray as the lattice point X_{ROI}, to T1, T2, . . . described above.

Here, in the case where the lattice points are set at equal intervals (Δt) on the time axis in the Y direction, the intervals are not necessarily equal intervals in terms of space. Accordingly, when calculating the time taken for the ultrasonic wave to reach each of the lattice points, a corrected Δt/2 may be used instead of Δt/2 in formula (3). Here, for example, the corrected Δt/2 is a value obtained by adding or subtracting to or from Δt/2 a value obtained by dividing the difference in depth (distance in the Y direction) between each of A1, A2, . . . and the lattice point An associated with the same sound ray as the lattice point X_{ROI}, by V. The depth of each of the lattice points A1, A2, . . . can be determined since the local sound velocity values in the lattice points at shallower depths are known.

In addition, the calculation of the virtual synthesized reception wave W_{SUM} is performed by superimposing default pulse waves (W_{A1}, W_{A2}, . . . ) emitted in practice from the lattice points A1, A2, . . . with the delays X_{ROI}A1/V, X_{ROI}A2/V, . . . .

Next, the error between the virtual reception wave W_{X} and the virtual synthesized reception wave W_{SUM} is calculated (step S68). The error between the virtual reception wave W_{X} and the virtual synthesized reception wave W_{SUM} is calculated by a method using cross-correlation therebetween, a method in which phase phasing addition is performed by multiplying the virtual reception wave W_{X} by a delay obtained from the virtual synthesized reception wave W_{SUM}, or a method in which phase phasing addition is performed by inversely multiplying the virtual synthesized reception wave W_{SUM} by a delay obtained from the virtual reception wave W_{X}. In order to obtain a delay from the virtual reception wave W_{X}, the lattice point X_{ROI} is set as the reflection point, and the time when the ultrasonic wave propagated at the sound velocity V reaches each of the elements may be taken as the delay. In addition, in order to obtain a delay from the virtual synthesized reception wave W_{SUM}, an equal phase line is extracted from the phase difference of the synthesized reception waves between adjacent elements and the equal phase line is set as the delay, or the phase difference at the maximum (peak) positions of the synthesized reception waves of each of the elements may simply be set as the delay. In addition, the cross-correlated peak positions of the synthesized reception waves from the elements may be set as the delay. The error during the phase phasing addition is determined by a method of using peak to peak of the waveform after the phasing addition or a method of using the maximum value of the amplitude after the envelope detection.

Next, when the calculation with all of the assumed sound velocity values is completed by repeating from step S64 to step S68 (“Y” in step S70), the local sound velocity value at the lattice point X_{ROI} is determined (step S72). In the case where the Huygens' principle is strictly applied, the waveform of the virtual synthesized reception wave W_{SUM} determined in step S66 described above is equal to the waveform of the virtual reception wave (reflected wave) W_{X} with the local sound velocity value at the lattice point X_{ROI} being assumed as V. In step S72, the value of the assumed sound velocity value where the difference between the virtual reception wave W_{X} and the virtual synthesized reception wave W_{SUM} is the minimum is determined to be the local sound velocity value at the lattice point X_{ROI}.

Instead of the methods described above (calculating the virtual synthesized reception waveform, calculating the error from the virtual reception waveform, and determining the sound velocity), a table may be used in which the ambient sound velocity value of the lattice point X_{ROI} and the ambient sound velocity values of lattice points A1, A2, . . . are inputs and the sound velocity value at the lattice point X_{ROI} is an output.

In addition, the determination of the local sound velocity value may be performed a plurality of times using lattice points with different intervals and different ranges.

The sound velocity map generator 27 stores the local sound velocity value determined by the local sound velocity determiner 25 in association with the positional information in the ultrasound image and generates a sound velocity map having the local sound velocity value and the positional information of the ultrasound image. The sound velocity map generator 27 supplies the information on the sound velocity map to the phasing addition section 38.

With this, when the reception focusing process is performed on the element data in the phasing addition section 38, it is possible to perform the reception focusing process based on the sound velocity map stored in the sound velocity map generator 27. In addition, when the transmission of the ultrasonic beam is performed by the transmission section 14, the delay amount of the driving signal may be adjusted based on the sound velocity map stored in the sound velocity map generator 27.

In the image generator 24, the phasing addition process is performed using the local sound velocity value determined by the local sound velocity determiner 25 and B-mode image data (display image data) to be displayed is generated. Then, the ultrasound image using the local sound velocity value is displayed on the monitor 28 as a moving image or a still image.

The sound velocity map generator 27 may be configured to sequentially update the local sound velocity value of a corresponding region every time the local sound velocity value is supplied from the local sound velocity determiner 25, or may be configured to generate a sound velocity map for every frame. In addition, as well as generating a sound velocity map for each frame, the sound velocity map generator 27 may store the sound velocity maps of previous frames up to a frame several frames before in addition to the latest sound velocity (sound velocity map).

The ultrasound diagnostic apparatus 10a can take ultrasound images in the motion picture photographing mode and the still picture photographing mode as with the ultrasound diagnostic apparatus 10 of the first embodiment and has the same effects as the ultrasound diagnostic apparatus 10 of the first embodiment.

With the ultrasound diagnostic apparatus 10a, it is also possible to carry out the multi-line process using the reception data after phasing addition in the same manner as the ultrasound diagnostic apparatus 10 of the first embodiment.

Next, description will be given of a third embodiment of the present invention.

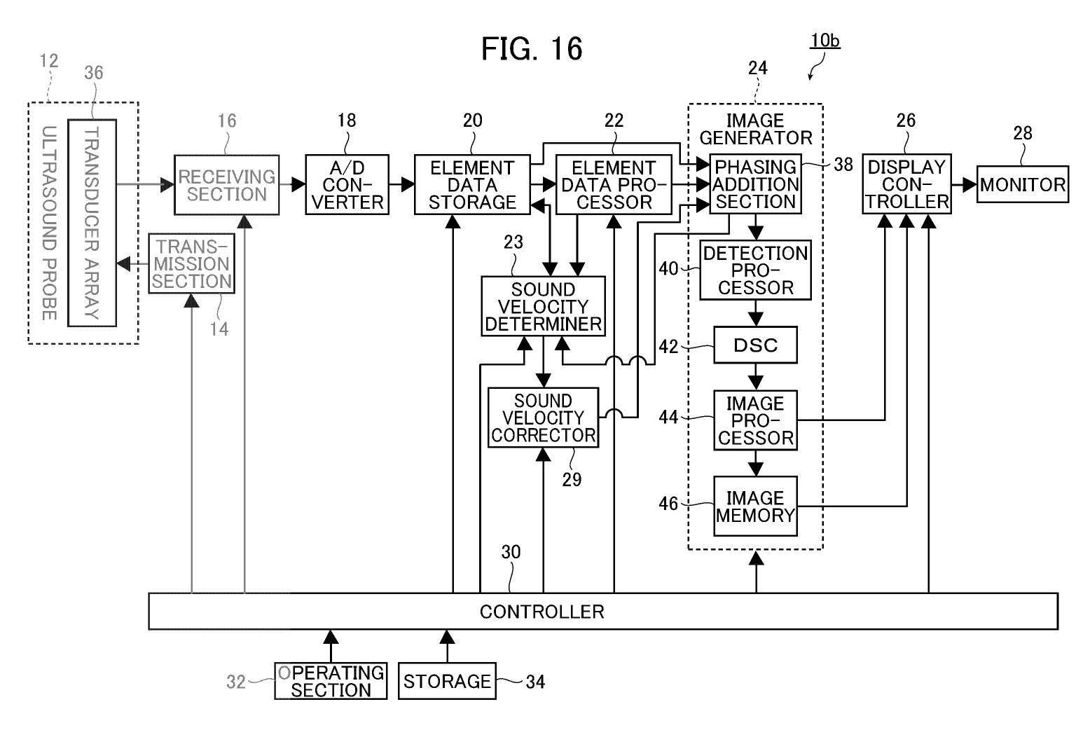

FIG. 16 is a block diagram illustrating another example of the ultrasound diagnostic apparatus of the embodiments of the present invention.

An ultrasound diagnostic apparatus 10b illustrated in FIG. 16 is different from the ultrasound diagnostic apparatus 10 illustrated in FIG. 1 in that a sound velocity corrector 29 is provided. Since the configuration is the same as that of the ultrasound diagnostic apparatus 10 illustrated in FIG. 1 in other respects, detailed description thereof will be omitted.

The sound velocity corrector 29 is connected with the sound velocity determiner 23 and the phasing addition section 38. The sound velocity corrector 29 is connected with the controller 30 and controlled by the controller 30.

The sound velocity corrector 29 corrects the sound velocity based on the ambient sound velocity and obtains, stores, and retains the sound velocity correction value. Specifically, the sound velocity corrector 29 replaces the initial set sound velocity with the calculated ambient sound velocity, and stores and retains the result. The initial set sound velocity is a sound velocity value set by default as the sound velocity value to be used in the generation of reception data in the phasing addition section 38.

The sound velocity correction value of the sound velocity corrector 29 is output to the phasing addition section 38. With this, when the reception focusing process is performed on the element data in the phasing addition section 38, it is possible to perform the reception focusing process based on the sound velocity correction value.

In the image generator 24, the phasing addition process is performed using the initial set sound velocity value which is set again by the sound velocity corrector 29, and B-mode image data (display image data) to be displayed is generated. Then, an ultrasound image where the sound velocity is corrected with the sound velocity correction value is displayed on the monitor 28 as a moving image or a still image.

The ultrasound diagnostic apparatus 10b can take ultrasound images in the motion picture photographing mode and the still picture photographing mode as with the ultrasound diagnostic apparatus 10 of the first embodiment and has the same effects as the ultrasound diagnostic apparatus 10 of the first embodiment.

With the ultrasound diagnostic apparatus 10b, it is also possible to carry out the multi-line process using reception data after phasing addition in the same manner as the ultrasound diagnostic apparatus 10 of the first embodiment.

The present invention is basically configured as described above. Moreover, detailed description has been given of the ultrasound diagnostic apparatus, the ultrasound image generating method, and the computer readable recording medium having stored therein the program of the present invention; however, the present invention is not limited to the embodiments described above and various improvements and modifications may be made within a range which does not depart from the gist of the present invention.

What is claimed is:

1. An ultrasound diagnostic apparatus inspecting an inspection object using ultrasonic beams, comprising:
a probe having a plurality of elements arranged therein, the probe being configured to transmit the ultrasonic beams, receive ultrasonic echoes reflected by the inspection object, and output analog element signals according to the received ultrasonic echoes;
a transmitter configured to cause the probe to transmit the ultrasonic beams plural times through the plurality of elements such that predetermined transmission focus points are formed;
a receiver configured to receive analog element signals that the plurality of elements output in response to transmission of each of the ultrasonic beams for each of the transmission focus points, and carry out a predetermined process;
an analog-to-digital converter configured to analog-to-digital convert the analog element signals processed by the receiver into pieces of first element data which are digital element signals;
a first data processor configured to generate a piece of second element data corresponding to one of the pieces of first element data from the pieces of first element data; and
a photographing mode switching unit configured to switch a mode between a motion picture photographing mode in which a moving image is taken by generating the ultrasonic beams continuously in terms of time and a still picture photographing mode in which a still image is taken by temporarily generating the ultrasonic beams,
wherein when the photographing mode switching unit switches the mode to the motion picture photographing mode, the transmitter forms at least one focus point in the inspection object, and the first data processor processes the pieces of first element data.

2. The ultrasound diagnostic apparatus according to claim 1, wherein the transmitter transmits the ultrasonic beams plural times while changing an element being center.

3. The ultrasound diagnostic apparatus according to claim 1, wherein the receiver changes an element being center in response to transmission of each of the ultrasonic beams by the transmitter.

4. The ultrasound diagnostic apparatus according to claim 1, wherein the receiver carries out reception using same elements as the plurality of elements used by the transmitter.

5. The ultrasound diagnostic apparatus according to claim 1, wherein the first data processor changes a number of the pieces of first element data to be processed when the photographing mode switching unit switches the mode to the motion picture photographing mode.

6. The ultrasound diagnostic apparatus according to claim 1, further comprising: an image generator configured to generate display image data based on the piece of second element data; and a monitor configured to display a moving image of an ultrasound image based on the display image data.

7. The ultrasound diagnostic apparatus according to claim 6, further comprising: an ambient sound velocity determiner configured to determine an ambient sound velocity in the inspection object,
wherein the image generator generates display image data using the determined ambient sound velocity, and
wherein the monitor displays a moving image of an ultrasound image based on the ambient sound velocity.

8. The ultrasound diagnostic apparatus according to claim 7, further comprising: a local sound velocity determiner configured to determine a local sound velocity based on the ambient sound velocity,
wherein the image generator generates the display image data using the determined local sound velocity, and
wherein the monitor displays a moving image of an ultrasound image based on the local sound velocity.

9. The ultrasound diagnostic apparatus according to claim 7, further comprising: a sound velocity corrector configured to correct a sound velocity based on the ambient sound velocity to obtain a sound velocity correction value,
wherein the image generator generates the display image data using the sound velocity correction value, and
wherein the monitor displays a moving image of an ultrasound image with a sound velocity having been corrected with the sound velocity correction value.

10. The ultrasound diagnostic apparatus according to claim 1, further comprising: a second data processor configured to generate data of one line on an ultrasound image based on one of the pieces of first element data,
wherein when the photographing mode switching unit switches the mode to the still picture photographing mode, the transmitter forms a plurality of focus points in the inspection object, and the second data processor processes the pieces of first element data.

11. The ultrasound diagnostic apparatus according to claim 10, further comprising: an image generator configured to generate display image data based on data of one line on an ultrasound image generated by the second data processor; and a monitor configured to display a still image of an ultrasound image based on the display image data.

12. The ultrasound diagnostic apparatus according to claim 11, further comprising: an ambient sound velocity determiner configured to determine an ambient sound velocity in the inspection object,
wherein the image generator generates display image data using an ambient sound velocity determined by the ambient sound velocity determiner, and
wherein the monitor displays a still image of an ultrasound image based on the ambient sound velocity.

13. The ultrasound diagnostic apparatus according to claim 12, further comprising: a local sound velocity determiner configured to determine a local sound velocity based on the ambient sound velocity,
wherein the local sound velocity determiner determines a local sound velocity based on the ambient sound velocity,
wherein the image generator generates display image data using the determined local sound velocity, and
wherein the monitor displays a still image of an ultrasound image based on the local sound velocity.

14. The ultrasound diagnostic apparatus according to claim 11, further comprising: a sound velocity corrector configured to correct a sound velocity based on the ambient sound velocity to obtain a sound velocity correction value,
wherein the image generator generates display image data using the sound velocity correction value, and
wherein the monitor displays a still image of an ultrasound image based on the sound velocity correction value.

15. The ultrasound diagnostic apparatus according to claim 1, further comprising: an element data retaining unit configured to retain at least either one of the pieces of first element data and the pieces of second element data.

16. The ultrasound diagnostic apparatus according to claim 1, wherein the first data processor generates pieces of first reception data by performing phasing addition on the respective pieces of first element data just before generating the piece of second element data from the pieces of first element data, and generates a piece of second reception data corresponding to one of the pieces of first reception data from the pieces of first reception data.

17. The ultrasound diagnostic apparatus according to claim 16, further comprising: an image generator configured to generate display image data based on the piece of second reception data; and a monitor configured to display a moving image of an ultrasound image based on the display image data.

18. The ultrasound diagnostic apparatus according to claim 1, wherein the first data processor includes a superimposition processor configured to generate the piece of second element data by superimposing two or more of the pieces of first element data based on receiving times when the plurality of elements receive ultrasonic echoes and positions of the plurality of elements.

19. An ultrasound diagnostic apparatus inspecting an inspection object using ultrasonic beams, comprising:
a probe having a plurality of elements arranged therein, the probe being configured to transmit the ultrasonic beams, receive ultrasonic echoes reflected by the inspection object, and output analog element signals according to the received ultrasonic echoes;
a transmitter configured to cause the probe to transmit the ultrasonic beams plural times through the plurality of elements such that predetermined transmission focus points are formed;
a receiver configured to receive analog element signals that the plurality of elements output in response to transmission of each of the ultrasonic beams for each of the transmission focus points, and carry out a predetermined process;
an analog-to-digital converter configured to analog-to-digital convert the analog element signals processed by the receiver into pieces of first element data which are digital element signals;
a first data processor configured to carry out a phasing addition process on the pieces of first element data and generate a piece of second element data corresponding to one of the pieces of first element data after phasing addition; and
a photographing mode switching unit configured to switch a mode between a motion picture photographing mode in which a moving image is taken by generating the ultrasonic beams continuously in terms of time and a still picture photographing mode in which a still image is taken by temporarily generating the ultrasonic beams,
wherein when the photographing mode switching unit switches the mode to the motion picture photographing mode, the transmitter forms at least one focus point in the inspection object, and first data processor processes the pieces of first element data after phasing addition.

20. An ultrasound image generating method for acquiring an ultrasound image for use in inspecting an inspection object using a probe having a plurality of elements arranged therein, the probe transmitting ultrasonic beams, receiving ultrasonic echoes reflected by the inspection object, and outputting analog element signals according to the received ultrasonic echoes, the method comprising the steps of:
when a mode is switchable between a motion picture photographing mode in which a moving image is taken by generating ultrasonic beams continuously in terms of time and a still picture photographing mode in which a still image is taken by temporarily generating the ultrasonic beams, and the mode is switched to the motion picture photographing mode,
causing the probe to transmit the ultrasonic beams plural times through the plurality of elements such that predetermined transmission focusing points are formed, while outputting analog element signals that the plurality of elements output in response to transmission of each of the ultrasonic beams; analog-to-digital converting the analog element signals into pieces of first element data which are digital element signals; and generating a piece of second element data corresponding to one of the pieces of first element data from the pieces of first element data,
with at least one focus point being formed in the inspection object.

21. The ultrasound image generating method according to claim 20, further comprising the steps of: forming a plurality of focus points in the inspection object; transmitting the ultrasonic beams to transmission focus points; obtaining the pieces of first element data; and generating data of one line on an ultrasound image based on one of the pieces of first element data when the mode is switched to the still picture photographing mode.

22. A computer readable recording medium having stored therein a program that causes a computer to execute the steps of the ultrasound image generating method according to claim 20 as a procedure.
